(19) United States
(12) Patent Application Publication
(10) Pub. No.: US 2002/0028482 A1
(43) Pub. Date: Mar. 7, 2002

(54) NIP45 HUMAN HOMOLOG

(76) Inventors: HONG ZHOU, WILMINGTON, DE (US); JIUQIAO ZHOAO, HOCKESSIN, DE (US); DERONG LIU, WILMINGTON, DE (US)

Correspondence Address: LIPD DEPT (FOC 1 S/E), ZENECA INC, 1800 CONCORD PIKE, P O BOX 5437, WILMINGTON, DE, 198505437

(21) Appl. No.: 09/175,254

(22) Filed: Oct. 20, 1998

(30) Foreign Application Priority Data
Oct. 24, 1997 (GB) GB9722388.7

(51) Int. Cl. C07H 21/04; C12P 21/06; C12N 15/00; C12N 15/09; C12N 15/63; C12N 15/70; C12N 15/74; C12N 5/00; C12N 5/02; C07K 1/00; C07K 14/00; C07K 17/00; C07K 16/00; A61K 7/46; G01N 33/53

(52) U.S. Cl. 435/69.1; 536/23.5; 435/320.1; 435/325; 530/350; 530/387.1; 435/7.1; 512/12

(57) ABSTRACT

A human NIP45 polypeptide is described which trans-activates transcription of the human IL-4 gene. A full length cDNA which encodes the novel trans-activator polypeptide is disclosed as well as the interior structural region and the amino acid residue sequence of the human NIP45. Methods are provided to identify compounds that modulate the biological activity of the native IL-4 transcription associated biomolecule and hence regulate IL-4 transcription.

Priority is claimed under 35 USC §119(a) from UK Application GB 9722388.7 entitled NIP45 HUMAN HOMOLOG, filed Oct. 24, 1997; the entire disclosure of which is incorporated by reference.

### FIELD OF THE INVENTION

The present invention relates to nucleic acid and amino acid sequences of a novel human NIP45 and to the use of these sequences to identify compounds that modulate the transcriptional activation activity of the native biomolecule. The invention is also related to the diagnosis, study, prevention, and treatment of pathophysiological disorders related to the biological molecule.

### BACKGROUND OF THE INVENTION

Cytokines coordinate a number of interactions between different cell types in multicellular organisms and play a major role in orchestrating the immune response. Production of cytokines is tightly controlled at several levels, transcription, translation, secretion, and, sometimes, activation of a precursor. Inappropriate cytokine production is involved in the pathogenesis of autoimmune and malignant diseases, as well as acute and chronic infections. The importance of cytokine production in a myriad of disease processes is now widely recognized. Cytokines and cytokine antagonists are recognized to have important roles in controlling the type of immune response generated. These mediators have the most profound effects if used at the initiation of an immune response. The control of IL-4 production, for example, has clear implications for immune manipulation for established autoimmune diseases. Seder, R.A., et al., Are differentiated human T helper cells reversible, International Archives of Allergy & Immunology, 113(1-3):163 (1997).

IL-4 has been called the “prototypic immunoregulatory cytokine.” Like many cytokines, it can affect a variety of target cells in multiple ways. IL-4 has an important role in regulating antibody production, hematopoiesis and inflammation, and the development of effector T-cell responses. Moreover, IL-4 is the major inducer of B-cell switching to IgE production and is therefore a key initiator of IgE-dependent, mast-cell-mediated reactions. In view of the clear correlation of aberrant expression with disease, it is of interest to understand the signals that regulate IL-4 expression in a cell-specific manner. Brown, M. A., et al., Functions of IL-4 and control of its expression, Critical Reviews in Immunology, 17(1):1 (1997); Abbas, A. K., et al., Nature 383:787 (1996).

Over the past decade, the central role of T-cells in the process of collagen-induced arthritis (CIA) has been extensively documented. The inflammatory features of CIA have been recently demonstrated by CD4+T-cell response shifting in vivo from a dominant Th0/Th1 response to a clear Th2 phenotype. Doncarli, A., et al., Conversion in vivo from an early dominant Th0/Th1 response to a Th2 phenotype during the development of collagen-induced arthritis, European Journal of Immunology, 27(6):1451 (1997). Autoreactive T-lymphocytes in peripheral blood of patients with rheumatoid arhritis, for example, dramatically increases relative to the levels in healthy individuals. Similarly, peripheral blood T-lymphocytes from patients with inflammatory arthritis proliferate strongly in the absence of exogenous antigen or mitogen. Welch, W. J., et al., The Stress Response and the Immune System, Inflammation: Basic Principles and Clinical Correlates, Raven Press, Gallin, J. I., et al., Eds., Second Edition, Chapter 41, 841 (1992). Chronic inflammatory disease, including rheumatoid arthritis, for instance, is believed to be mediated by activated T-lymphocytes that infiltrate the synovial membrane and initiate a series of inflammatory processes. Panayi, G. S., et al., The Importance of the T-Cell in Initiating and Maintaining the Chronic Synovitis of Rheumatoid Arthritis, Arthritis Rheum, 35:729 (1992).

The existence of subsets of T-cells that differ in their cytokine secretion patterns and effector functions provides a framework for understanding the heterogeneity of normal and pathological immune responses. Defining the cellular and molecular mechanisms of helper- T-Cell differentiation is expected lead to rational strategies for manipulating immune responses for prophylaxis and therapy. Abbas, A. K., et al., Nature 383:787 (1996).

Accumulating evidence also indicates that the autoimmune disease multiple sclerosis (MS) is mediated by autoreactive T-lymphocytes. Stinissen, P., et al., Crit. Rev. Immunol., 17(1):33 (1997). Serum IL-4 levels have been recently demonstrated to be significantly higher in patients with systemic sclerosis than in the controls. Hasegawa M., et al., Journal of Rheumatology. 24(2):328 (1997). Autoreactive T-lymphocytes have been demonstrated to undergo in vivo activation and clonal expansion in patients with MS. Zhang, J., et al., J. Mol. Med., 74(11):653 (1996).

Another propelling recent development in the implication of overactive T-cells is the recognition that a particular subset of T-lymphocytes appear to be a major culprit in asthma and other allergic diseases, by responding with undue vigor to apparently harmless invaders (rates of asthma per capita in the developing world have increased dramatically in the last several decades; doubling in the U.S. since 1980). New Clues to Asthma Therapies: Vogel, G., Science, 276:1643 (1997). IL-4 is expressed almost universally in the nasal mucosa of patients with perennial allergic rhinitis during natural allergen exposure and has been implicated to play a crucial role in the development of allergic nasal diseases in vivo. Lee, C. H., et al., Annals of Otology, Rhinology & Laryngology, 106(3):215 (1997).

Evidence has demonstrated IL-4 gene expression is responsible for triggering biological effects across a wide variety of pathophysiological conditions including conditions manifested by dysfunctional leukocytes, T-lymphocytes, e.g. acute and chronic inflammatory disease, auto-immune disorders, rheumatoid arthritis, myasthenia gravis, transplant rejection, asthma, Hodgkin's disease, and allergic response.

Transcriptional regulation, including transcriptional trans-activation factors, of the IL-4 gene has been studied by various groups in the last several years. The region from −100 to −28 relative to the transcription start site of the IL-4 promoter has been shown to be sufficient to confer inducible expression. Ho, I-Cheng, et al., The Proto-Oncogene c-maf is Responsible for Tissue-Specific Expression of Interleukin-4, Cell, 85:973 (1996). AP-1, NFAT and MARE cis regulatory elements have been found in this region. AP-1, NFATp and c-maf proteins have been shown to bind to these elements. The induction of IL-4 gene transcription is known to be mediated in part by the nuclear factor of activated T cells (NF-AT). Transactivators involved in the mechanisms of NF-AT-mediated transcription have heretofore been relatively unknown. Recently, Hodge et al identified a nuclear factor that interacts with the Rel homology domain (RHD) of NF-ATp factor. Science, 274:1903 (1996). The murine isolate, designated NIP45 (for NF-ATp interacting protein) has minimal similarity to any known gene. NIP45 has been shown function as an integral part of a cistron with transcriptional associated biomolecules NF-ATp and the proto-oncogene c-Maf to activate the interleukin-4 (IL-4) cytokine promoter. Transient overexpression of NIP45 with NF-ATp and c-Maf in B lymphoma cells induces endogenous IL-4 protein production. NIP45 has also been demonstrated, in combination with NF-ATp and c-Maf, to activate the IL-4 gene promoter in vitro. See, e.g., Rao, A., et al., NFATp, A cyclosporin-sensitive transcription factor implicated in cytokine gene induction, J. Leukocyte Biology 57:536 (1995); Ho, IC, et al., The proto-oncogene c-maf is responsible for tissue-specific expression of interleukine-4, Cell 85:973 (1996); Peltz, G., Transcription factors in immune-mediated disease, Current Opinion in Biotechnology 8:467 (1997); Vogel G., New Clues to Asthma Therapies, Science 276:1643 (1997); Abbas, A. K., et al., Nature 383:787 (1996); Nakamura, T., et al., J. Immunology 158:2648 (1997).

Transcription factors have become more and more attractive generally as drug targets due to the inherent value of the ability to specifically control target effector genes. IL4 is the known primary driving force in the differentiation of Th0 to Th2 helper cells. Compounds which specifically disrupt the interaction between transcriptional activators and their substrate in the IL-4 cistron are strongly expected to have significant value inter alia as anti-inflammation drugs and/or drugs to treat auto-immune disease. Thus, the ability to control a critical IL-4 transcriptional regulation factor is of paramount value toward anti-inflammation and immunosupressant drug development. Toward this end NIP45 appears to be a good candidate target for HTP screening and/or testing system for drugs which will alleviate T-cell dependent autoimmune and allergic responses; and for for cytokine-based therapies of chronic disease. However, the previously reported NIP45 is a murine isolate. The availability of a functional human homolog will be ideal for such drug screening and testing purposes.

### SUMMARY OF THE INVENTION

The present invention is directed to an isolated and purified polynucleotide molecule, which encodes a polypeptide of a human NIP45, or a biologically active derivative thereof comprising a nucleic acid sequence encoding the polypeptide having the sequence substantially as depicted in SEQ ID NO:3 or a biologically active fragment thereof. Isolated and purified polynucleotides of the present invention include but are not limited to SEQ ID NO:1 (novel human NlP45 cDNA) and SEQ ID NO:2 (novel human NIP45 structural coding region).

In addition, the current invention is directed to a purified polypeptide comprising the amino acid sequence substantially as depicted in SEQ ID NO:3 which functions as a human NIP45 transcriptional activator polypeptide.

The invention is further directed to an expression vector for expression of a novel human NIP45 polypeptide in a recombinant host cell, wherein said vector contains a polynucleotide comprising a nucleic acid sequence encoding a human trans-activator polypeptide having the sequence substantially as depicted in SEQ ID NO:3 or a biologically active derivative thereof.

Further the invention is directed to a host cell containing an expression vector for expression of a novel human NIP45 polypeptide, wherein said vector contains a polynucleotide comprising a nucleic acid sequence encoding the polypeptide of a human NIP45 having the sequence substantially as depicted in SEQ ID NO:3 or a biologically active derivative thereof. The invention is also directed to a method for producing a polypeptide wich has the ability to trans-activate IL-4 transcription having the amino acid sequence substantially as depicted in SEQ ID NO:3 by culturing said host cell under conditions suitable for the expression of said polypeptide, and recovering said polypeptide from the host cell culture.

The instant invention is further directed to a method of identifying compounds that modulate the biological activity of a human NIP45, comprising:

(a) combining a candidate compound modulator of human NIP45 biological activity with a NIP45 polypeptide comprising the sequence substantially as depicted in SEQ ID NO:3, and

(b) measuring an effect of the candidate compound modulator on the biological activity.

The instant invention is further directed to a method of identifying compounds that modulate the biological activity of a human NIP45, comprising:

(a) combining a candidate compound modulator of human NIP45 biological activity with a host-cell expressing a NIP45 polypeptide comprising the sequence substantially as depicted in SEQ ID NO:3, and

(b) measuring an effect of the candidate compound modulator on the biological activity.

The instant invention is further directed to a method of identifying compounds that modulate the transcriptional activation of IL-4, comprising:

(a) combining a candidate compound modulator of transcriptional activation of IL-4 with a polypeptide of a human trans-activator having the sequence substantially as depicted in SEQ ID NO:3, and an IL-4 cistron, and

(b) measuring an effect of the candidate compound modulator on the transcriptional activation of the IL-4 cistron.

The instant invention is further directed to a method of identifying compounds that modulate the transcriptional activation of IL-4, comprising:

(a) combining a candidate compound modulator of transcriptional activation of IL-4 with a host-cell comprising an IL-4 cistron and expressing a polypeptide of a human trans-activator having the sequence substantially as depicted in SEQ ID NO:3, and

(b) measuring an effect of the candidate compound modulator on the transcriptional activation of the IL-4 cistron.

The present invention is also directed to active compounds identified by means of the aforementioned methods, wherein said compounds modulate the biological activity of a human NIP45.

The present invention is also directed to active compounds identified by means of the aforementioned methods, wherein said compounds modulate the transcriptional activation of IL-4.

Further, the invention is directed to a pharmaceutical composition comprising a compound active in at least one of the aforementioned methods, wherein said compound is a modulator of the biological activity of a human NIP45.

Further, the invention is directed to a pharmaceutical composition comprising a compound active in at least one of the aforementioned methods, wherein said compound is a modulator of the transcriptional activation of IL-4.

Additionally, the invention is directed to a novel treatment of a patient in need of such treatment for a condition which is mediated by IL-4 gene expression, or for a condition which is mediated by the biological activity of human NIP45, comprising administration of a human NIP45 modulating compound active in at least one of the aforementioned methods.

The invention is further directed to an antisense poynucleotide molecule comprising substantially the complement of SEQ ID NO:2 or a biologically-effective portion thereof as well as a method for inhibiting the expression of a human NIP45 trans-activator biological molecule comprising administering an effective amount of the antisense molecule.

The invention is further directed to an antisense poynucleotide molecule comprising substantially the complement of SEQ ID NO:2 or a biologically-effective portion thereof as well as a method for modulating the expression of IL-4 in a cell comprising administering an effective amount of the antisense molecule.

The current invention is also drawn toward an antibody specific for a purified polypeptide comprising the amino acid sequence substantially as depicted in SEQ ID NO:3.

The invention is also directed to various diagnostic composition polypeptide sequence comprising the amino acid sequence substantially as depicted in SEQ ID NO:3.

### BRIEF DESCRIPTION OF THE FIGURES

FIG. 1 displays SEQ ID NO:1 which is a 2576 base cDNA nucleic acid sequence which encodes the novel human NIP45 (IL-4 transcriptional trans-activator) polypeptide described herein.

FIG. 2 displays SEQ ID NO:2 which is the 1260 base translated structural region, ATG to TGA, of the cDNA nucleic acid sequence which encodes the novel human NIP45 polypeptide described herein.

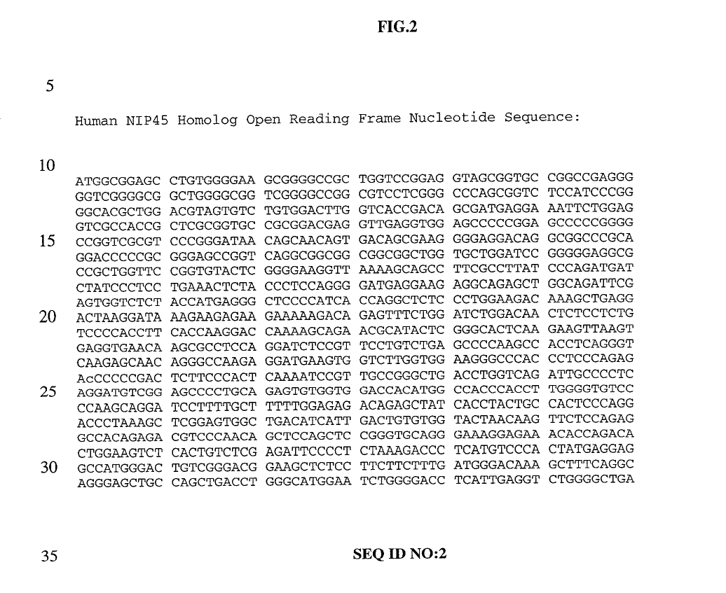

FIG. 3 displays SEQ ID NO:3 which is the 419 amino acid residue sequence of the novel human NIP45 polypeptide described herein.

FIG. 4 shows SEQ ID NO:4 which is the 412 amino acid residue sequence of the recently described murine NIP45. Hodge, M., et al., N-AT-Driven Interleukin-4 Transcription Potentiated by NIP45, Science, 274:1903 (1996).

FIG. 5 shows a comparison between the amino acid residue sequence of the novel human NIP45 polypeptide described herein (SEQ ID NO:3) (designated hNIP45), and the amino acid residue sequences of the recently described murine NIP45 polypeptide (SEQ ID NO:4).

Conserved amino acid residues are boxed. Dashes represent gaps introduced to optimize the alignment. Sequences shown in this figure were produced using the multisequence alignment program of DNASTAR software (DNASTAR Inc, Madison Wis.).

FIG. 6 shows the 5′-100 to −28 nucleic acid base positions of IL-4 promoter region relative to the transcription start site of the IL-4 gene which is sufficient to confer inducible expression. Ho, I-Cheng, et al., The Proto-Oncogene c-maf is Responsible for Tissue-Specific Expression of Interleukin-4, Cell, 85:973 (1996). AP-1, NFAT and MARE cis regulatory elements have been found in this region. AP-1, NFAp and c-maf proteins have been shown to bind to these elements. The “IL-4 cistron” refers to the IL-4 promoter sequence and reporter gene, which in a preferred embodiment is the IL-4 structural coding region, as well as transcription associated biomolecules including but not limited to transcriptional activators NFAT (NFATp and/or NFAT 1), c-Maf and/or h-Maf, and NIP45.

FIG. 7 illustrates the mechanism by which hNIP45, NFAT, and c-Maf regulate T-cell activation.

FIG. 8 illustrates yeast 2 hybrid mapping results wherein hNIP45, particularly the N-terminal portion, is demonstrated to interact with hNFAT1 (numerical values indicate hNIP45 SEQ ID NO:3 positions).

FIG. 9 illustrates the mechanism by which yeast two hybrid high throughput screening assays operate to identify hNIP45 antagonist compounds as described herein.

FIG. 10 illustrates a yeast two hybrid high throughput screening procedure to identify compounds which specifically modulate the activity of hNIP45.

FIG. 11 shows yeast two hybrid assay results which indicate mNIP45 (murine) interacts well with mNFATp (murine) (colonies in the top two rows), hNIP45 interacts well with hNFAT 1 (colonies in the bottom two rows) but slightly weaker than the interaction between mNIP45 and mNFATp, and that mNIP45 does not interact with hNFAT 1 very well (colonies in the middle two rows).

FIG. 12 further shows yeast two hybrid assay results which indicate mNIP45 (murine) interacts well with mNFATp (murine) (colonies in the top two rows), hNIP45 interacts well with hNFAT1 (colonies in the bottom two rows) but slightly weaker than the interaction between mNIP45 and mNFATp, and that mNIP45 does not interact with hNFAT 1 very well (colonies in the middle two rows).

### DETAILED DESCRIPTION OF THE INVENTION

Unless defined otherwise, all technical and scientific terms used herein have the same meaning as is commonly understood by one of skill in the art to which this invention belongs. All publications and patents referred to herein are incorporated by reference. Nucleic acid sequence as used herein refers to an oligonucleotide, nucleotide or polynucleotide sequence, and fragments or portions thereof, and to DNA or RNA of genomic or synthetic origin which may be double-stranded or single-stranded whether representing the sense or antisense strand. Similarly, amino acid and/or residue sequence as used herein refers to peptide or protein sequences or portions thereof.

Purified as used herein refers to molecules, either nucleic acid or amino acid sequences, that are removed from their natural environment and isolated or separated from at least one other component with which they are naturally associated.

“Transcription associated biomolecules” as used herein refer to factors that are directly or indirectly associated with transcriptional regulation including but not limited to transcriptional activators NFAT (NFATp and/or NFAT1), c-Maf, and NIP45.

“Biological activity” as used herein, in reference to hNIP45, refers to the ability of hNIP45 to interact with transcription associated biomolecules, including but not limited to, the ability of hNIP45 to interact with NFAT (NFATp and/or NFAT1), c-Maf.

An “IL-4 cistron” as used herein refers to an IL-4 promoter sequence and reporter gene, which in a preferred embodiment is the IL-4 structural coding region, as well as transcription associated biomolecules required for expression of the reporter gene.

Regulation of transcription as used herein refers to down regulation via antagonization, repression, neutralization, or sequestration, of a transcription associated biomolecule including but not limited to NIP45; as well as up regulation via transcriptional activation including but not limited to the biological activity of a NIP45 molecule described herein or agonization thereof by a compound identified by means described herein; as well as up regulation via antagonisation, neutralization, or sequestration of a repressor.

“Substantially as depicted” as used herein refers to functional derivative proteins, peptides and DNA sequences that may have changes but perform substantially the same biological function in substantially the same way; however, “substantially as depicted” is also intended to encompass dominant negative mutants versions of the hNIP45 described herein.

As used herein, a functional derivative of a human NIP45 molecule disclosed herein is a compound that possesses a biological activity (either functional or structural) that is substantially similar to SEQ ID NO:3. The term “functional derivatives” is intended to include the “fragments,” “variants,” “degenerate variants,” “analogs” and “homologues”, and to “chemical derivatives”. The term “variant” is meant to refer to a molecule substantially similar in structure and function to either an entire human NIP45 molecule or to a fragment thereof. A molecule is “substantially similar” to a NIP45 polypeptide if both molecules have substantially similar structures or if both molecules possess similar biological activity. The term “analog” refers to a molecule substantially similar in function to either an entire native transcriptional activator human NIP45 polypeptide, or to a fragment thereof.

Biologically active fragment as used herein includes peptides which have been truncated with respect to the N- or C-termini, or both; or the corresponding 5′ or 3′ end, or both, of the corresponding polynucleotide coding region, which fragments perform substantially the same biological function or encode peptides which perform substantially the same function as the precursor. The term “biologically active” also refers to the activity of a homolog or analog entity having structural, regulatory or biochemical functions substantially the same as the naturally occurring entity.

The term “modulate” or “modulation” is used herein to refer to the capacity to either enhance, emulate or inhibit the biological activity or otherwise effect a functional property of human NIP45 of the present invention including enhancement or inhibition of IL-4 transcriptional activation.

Expression vector as used herein refers to nucleic acid vector constructions which have components to direct the expression of heterologous protein coding regions including coding regions of the present invention through accurate transcription and translation in host cells. Expression vectors usually contain a promoter to direct polymerases to transcribe the heterologous coding region, a cloning site at which to introduce the heterologous coding region, and usually polyadenylation signals. Expression vectors include but are not limited to plasmids, retroviral vectors, viral and synthetic vectors.

Transformed host cells as used herein refer to cells which have coding regions of the present invention stably integrated into their genome, or episomally present as replicating or nonreplicating entities in the form of linear nucleic acid or transcript or circular plasmid or vector.

Direct administration as used herein refers to the direct administration of nucleic acid constructs which encode reagents (e.g., hNIP45, modulator compound molecule, antisense molecule, antibody molecule) of the present invention or fragments thereof; and the direct administration of reagents of the present invention or fragments thereof, per se; and the in vivo introduction of gene fusions of the present invention preferably via an effective eukaryotic expression vector in a suitable pharmaceutical carrier. Gene fusions of the present invention may also be delivered in the form of nucleic acid transcripts.

The transcription factors that regulate the expression of an increasing number of genes involved in the immune response have been identified and characterized. This information has led to the belief that therapeutic agents targeting these transcription factors can be developed. The clinical utility of cyclosporin A, an immunosuppressive agent that inhibits a transcription factor in T-lymphocytes demonstrates the potential value of transcription factor inhibitors as pharmaceutical agents. Moreover, glucocorticoids, which are widely used as anti-inflammatory agents, inhibit NF-κB-dependent gene transcription induced by inflammatory mediators. Peltz, G., Transcription factors in immune-mediated disease, Current Opinion in Biotechnology 8:467 (1997).

Cis- and trans-cistron requirements for IL-4 Gene Activation

IL-4 is the known primary driving force in the differentiation of Th0 to Th2 helper cells, which are found to be the major cellular mediator of clinical diseases such as Asthma, Allergy and many other Autoimmune Disorders. Thus, the IL-4 gene and integral components which regulate gene expression are currently a well recognized targets for anti-inflammation drug development. Three interacting proteins have recently been shown to contribute to expression of the IL-4 gene: NFAT, c-MAF, and NIP45.

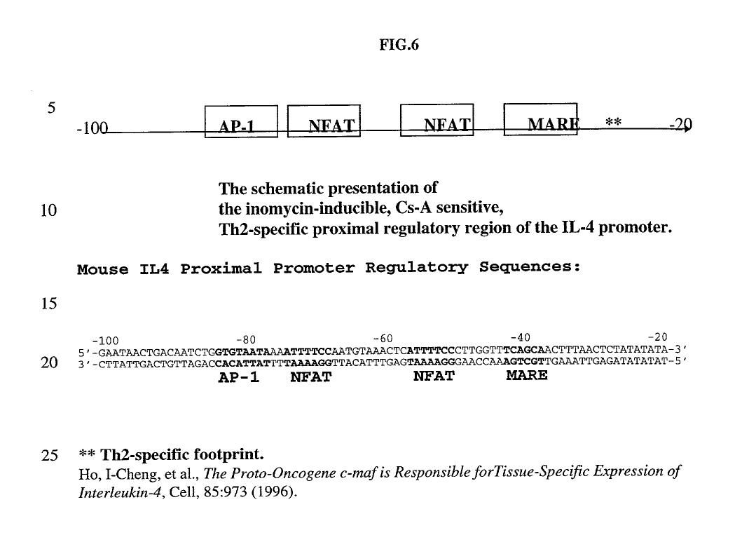

The 5′−100 to −28 nucleic acid base positions of IL-4 promoter region relative to the transcription start site of the IL-4 gene (FIG. 6) has been shown to be sufficient to confer inducible expression in response to ionomycin or cross-linking of the TCR in a Th2 cell line. Ho, I-Cheng, et al., The Proto-Oncogene c-maf is Responsible for Tissue-Specific Expression of Interleukin-4, Cell, 85:973 (1996). AP-1, NFAT and MARE cis regulatory elements have been found in this region. AP-1, NFATp and c-maf proteins have been shown to bind to these elements.

NFATp is expressed in several types of immune cells as a cytosolic protein that translocates to the nucleus following activation. The nuclear translocation is regulated by calcium and calcineurin and inhibited by cyclosporin A and FK506. In the nucleus, NFATp cooperates with Fos-Jun dimers and other transcription factors at composite elements in the regulatory regions of cytokine genes. AP-1 complexes are composed of homodimers or heterodimers of fos, jun transcription factors. Rao, A., et al., NFATp, A cyclosporin-sensitive transcription factor implicated in cytokine gene induction, J. Leukocyte Biology 57:536 (1995). NFATp(murine)/NFAT1(human) is a cytosolic protein of (120 kD). NFATp/NFAT1 possesses two transactivation domains whose sequences are not conserved in the other NFAT-family proteins, and a conserved DNA-binding domain that mediates the recruitment of cooperating nuclear transcription factors even when it is expressed in the absence of other regions of the protein. When expressed in COS cells, however, NFAT1 is capable of transactivation. CsA treatment of Raji B and Jurkat T cell lines yields a phosphorylated form of NFATp that is inhibited in DNA-binding and in its ability to form an NFAT complex with Fos and Jun. Dephosphorylation by in vitro treatment with calcineurin or alkaline phosphatase restores NFATp DNA binding activity and its ability to reconstitute an NFAT complex with Fos and Jun proteins. U.S. Pat. No. 5,656,452, NF-AT[p], A T-lymphocyte DNA-Binding Protein, issued Aug. 12, 1997—and all methods therein described—are herein incorporated by reference (Plasmid hNF-AT[p]21B2 containing the human NF-AT[p ]sequence and the plasmid mNF-AT[p]-Q1B1/A containing the murine NF-AT[p] are 3available from the American Type Culture Collection, Rockville, Md., having ATCC designations 75598 and 75597, respectively).

The proto-oncogene c-maf, a basic region/leucine zipper transcription factor, controls tissue-specific expression of IL-4. c-Maf is expressed in Th2 but not Th1 clones and is induced during normal precursor cell differentiation along a Th2 but not Th1 lineage. c-Maf binds to a c-Maf response element (MARE) in the proximal IL-4 promoter-adjacent to a site footprinted by extracts from Th2 but not Th1 clones. The tissue-specific IL-4 proximal promoter possesses NFAT and c-maf (MARE) binding sites. See, e.g., FIG. 6. Ectopic expression of c-Maf transactivates the IL-4 promoter in Th1 cells, B cells, and nonlymphoid cells, a function that maps to the MARE and Th2-specific footprint. Furthermore, c-Maf acts with the nuclear factor of activated T cells (NF-ATp) to initiate endogeneous IL-4 production by B cells. Ho, I.C., et al., Cell, 85(7):973 (1996).

A 1.6-kilobase pair full-length cDNA encoding a transcription factor homologous to the Maf family of proteins has recently been described. The human native protein, which is designated hMAF, contains a basic DNA binding domain and an extended leucine zipper. Marini, M. G., et al., hMAF, a small human transcription factor that heterodimerizes specifically with Nrf1 and Nrf2, J. Biol Chem Jun 27, 1997; 272(26):16490 (1997). This hMAF is a preferred embodiment (analogous to c-maf) for use in screening methods of the present invention.

Recently, Hodge et al identified a nuclear factor that interacts with the Rel homology domain (RHD) of NF-ATp factor. Science, 274:1903 (1996). The murine isolate, designated NIP45 (for NF-ATp interacting protein) has minimal similarity to any known gene. NIP45 has been shown function as an integral part of a cistron with transcriptional associated biomolecules NF-ATp and the proto-oncogene c-Maf to activate the interleukin-4 (IL-₄) cytokine promoter. Transient overexpression of NIP45 with NF-ATp and c-maf in B lymphoma cells induces endogenous IL-4 protein production. NIP45 has also been demonstrated, in combination with NF-ATp and c-Maf, to activate the IL-4 gene promoter in vitro. Thus NIP45 substantially stimulates the ability of NF-AT to activate transcription of genes that contain binding sites for NF-AT. See, e.g., Rao, A., et al., NFATp, A cyclosporin-sensitive transcription factor implicated in cytokine gene induction, J. Leukocyte Biology 57:536 (1995); Ho, IC, et al., The proto-oncogene c-maf is responsible for tissue-specific expression of interleukine-4, Cell 85:973 (1996); Peltz, G., Transcription factors in immune-mediated disease, Current Opinion in Biotechnology 8:467 (1997). NIP45 is evenly distributed throughout the nucleus. Overexpression of murine NIP45 in a cellular assay system using HepG2 cells has been shown to result in a 200-fold increase of endogenous IL-4 production. Hodge, M., et al., NF-AT-Driven Interleukin-4 Transcription Potentiated by NIP45, Science, 274:1903 (1996). Intervention with a compound which deprives the IL-4 promoter of the NIP45 transactivating complex is expected lead to a significant decrease of IL-4 transcription.

Transient co-transfection of NIP45, c-Maf and MF-ATp into the B lymphoma cells induced a 50- to 200-fold increase in endogenous IL-4 protein production. This result raises the significant possibility that a human NIP45 may be the first transcription factor that selectively regulates cytokine (IL-4) production. Peltz, G., Transcription factors in immune-mediated disease, Current Opinion in Biotechnology 8:467 (1997).

Genetic linkage studies have demonstrated the relationship between asthma susceptibility and the binding of NFAT1 to the IL-4 promoter (Rosenwasser, et al., (1997). Expression of NIP-45 promotes and stabilizes this binding.

NIP45 may regulate IL-2-synthesis as well since regulation of IL-2 synthesis by activated NF-ATp is well established.

hNIP45

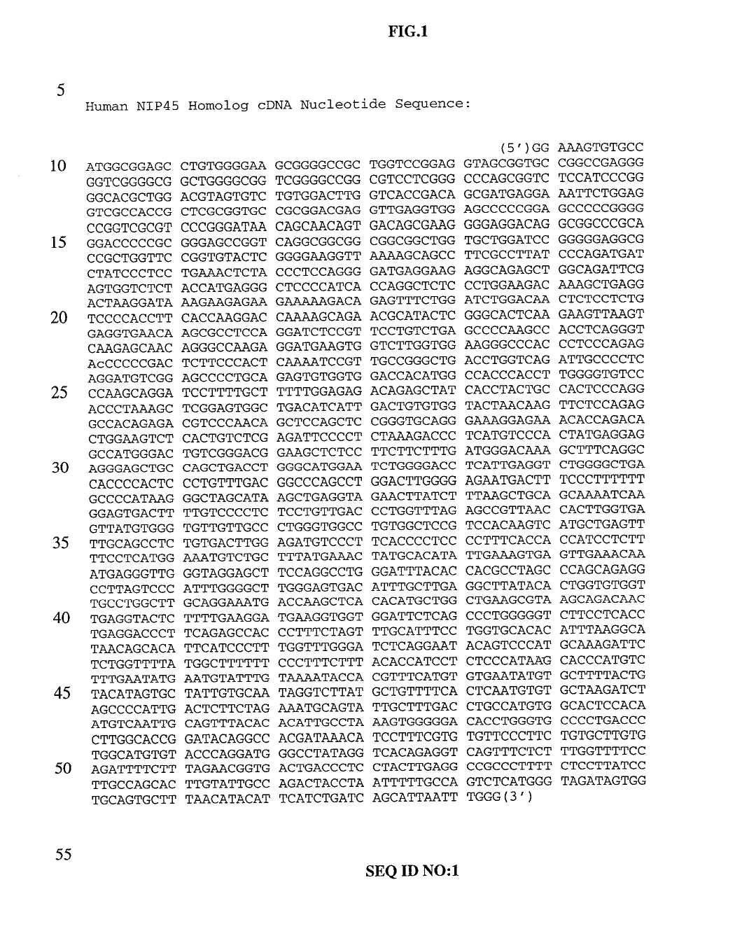

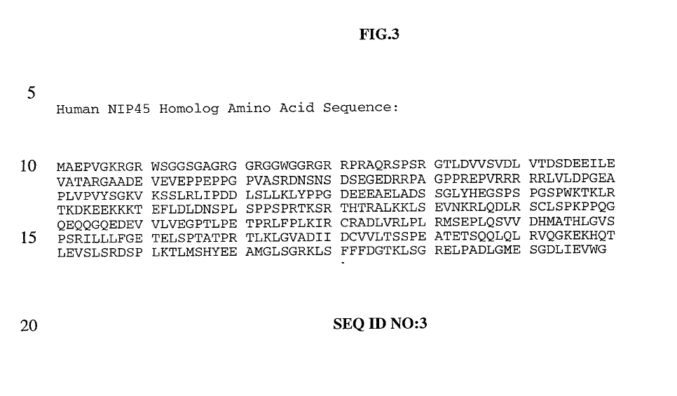

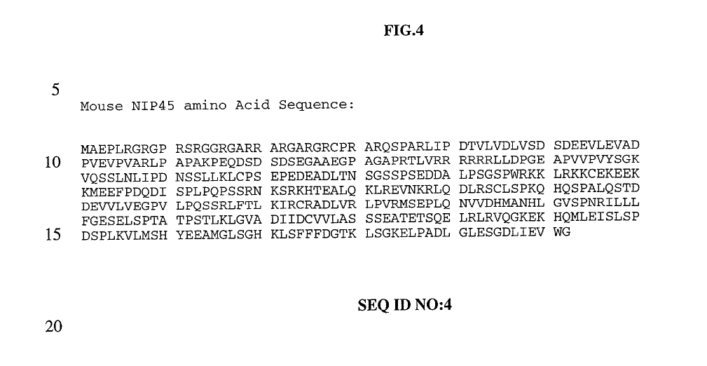

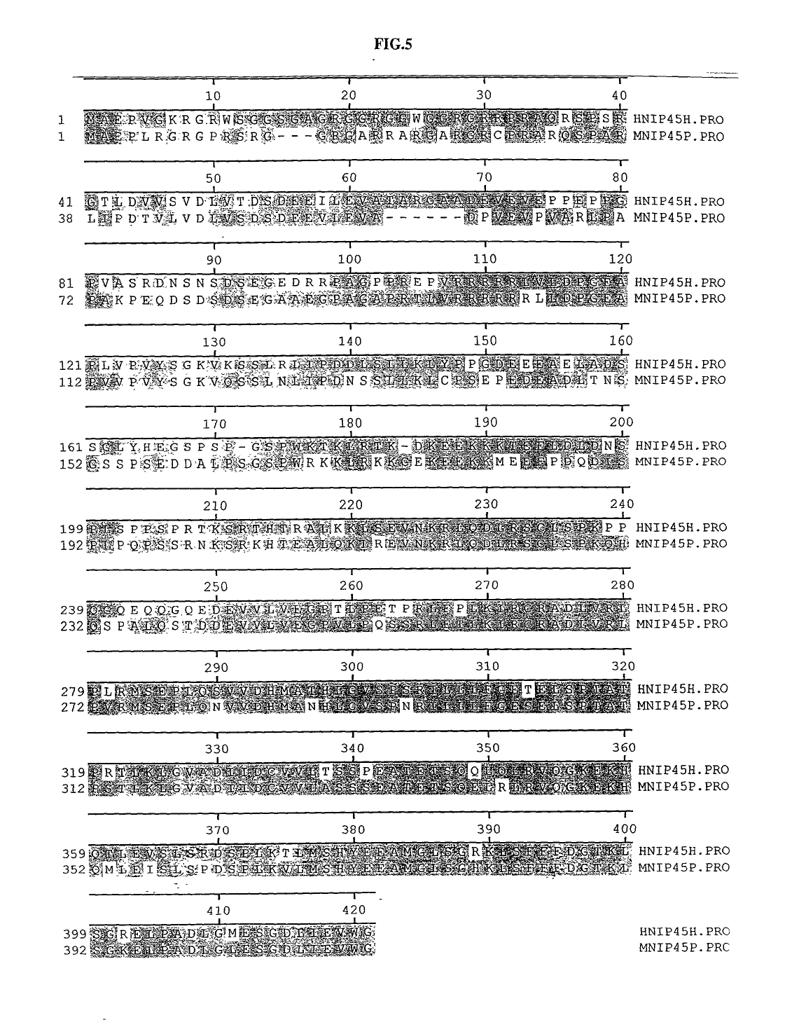

The open reading frame of the native human homologue of NIP45 is composed of 1260 nucleotides (SEQ ID NO:2) and codes for a peptide of 419 amino acid residues (SEQ ID NO:3) whereas the native murine NIP45 is composed of 412 amino acid residues (SEQ ID NO:4). The 2576 nucleotide cDNA sequence of hNIP45 (SEQ ID NO:1) is shown in FIG. 1. The homology at the amino acid residue level (SEQ ID NO:3, SEQ ID NO:4; see FIGS. 3-5) is about 80%. In contrast, the 3′ untranslated hNIP45 nucleic acid sequence is very different from the homolog of murine origin. The human homolog described herein clearly demonstrates high conservation in terms of the peptide size and coding region amino acid sequence. Protein sequence analyses indicates that these two peptides have similar native conformation; however, there is substantial biochemical divergence and hence significant pharmacological differences attributable to the biochemical characteristics of the phylogenetic diverse species from which they each originate. See, FIG. 5.

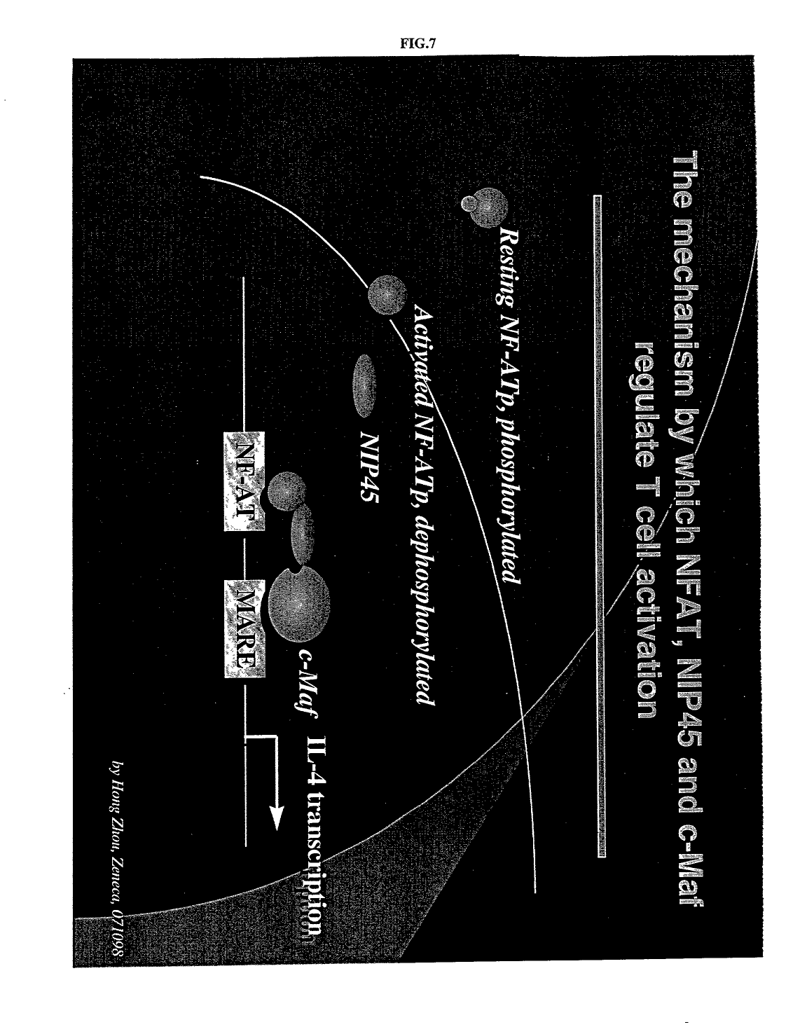

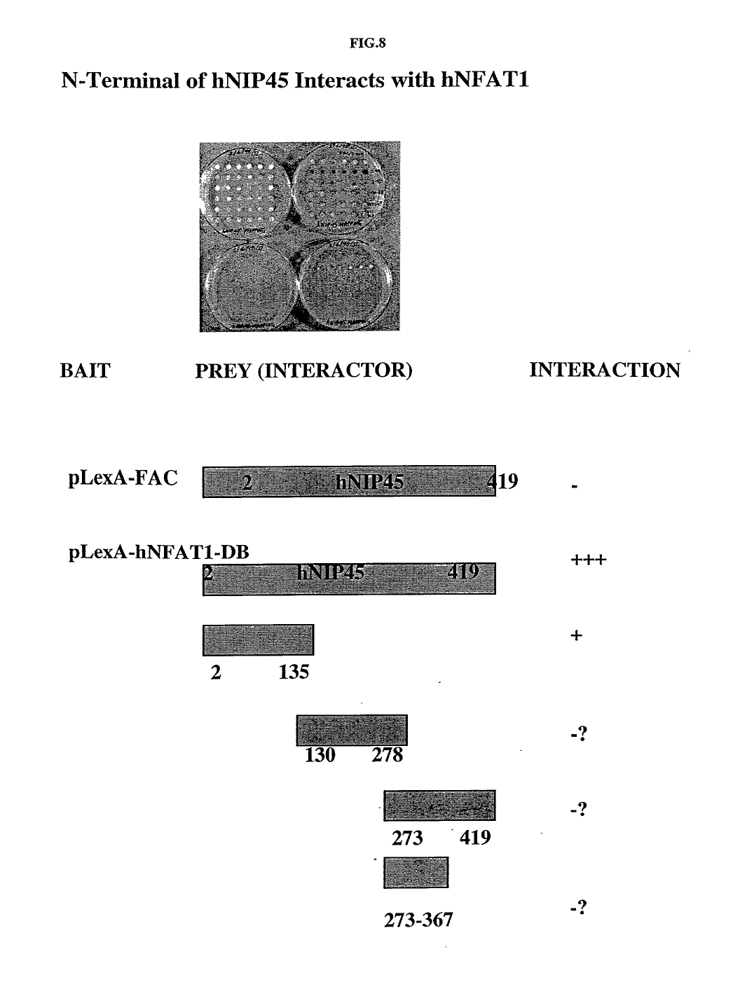

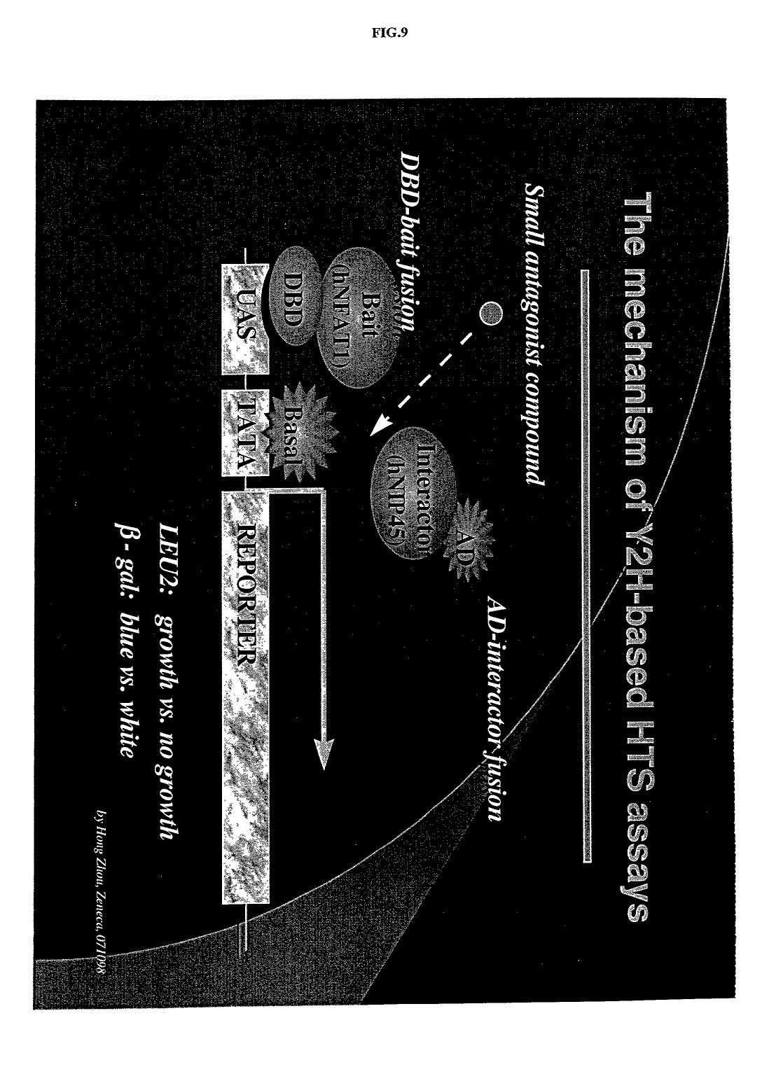

FIG. 7 illustrates the mechanism by which hNIP45, NFAT, and c-MAF regulate T-cell activation. FIG. 8 illustrates yeast 2 hybrid mapping results wherein hNIP45, particularly the N-terminal portion, is demonstrated to interact with hNFAT1 (numerical values indicate hNIP45 SEQ ID NO:3 positions). This experiment demonstrated that hNIP45 indeed interacts with hNFAT1, and the interaction domain of hNFAT1 likely overlaps with its DNA binding domain. See, Example VI. The yeast two hybrid system is also used for high throuput screening described herein. See, Example VIII, FIG. 9.

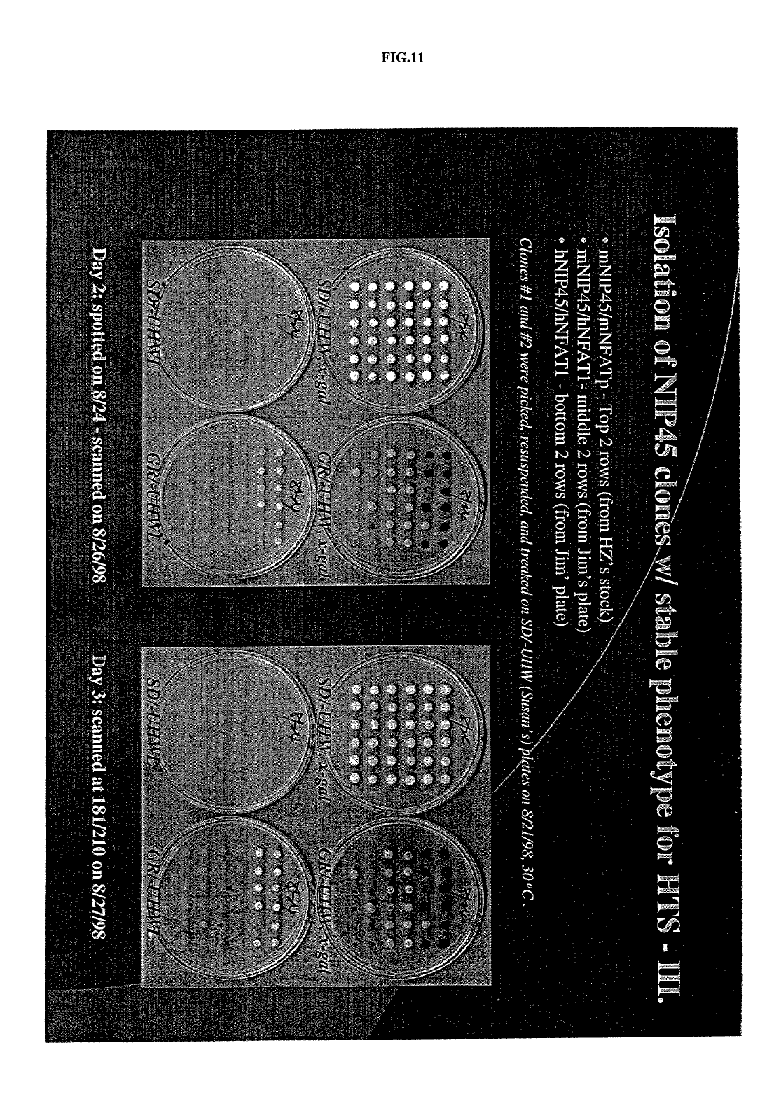

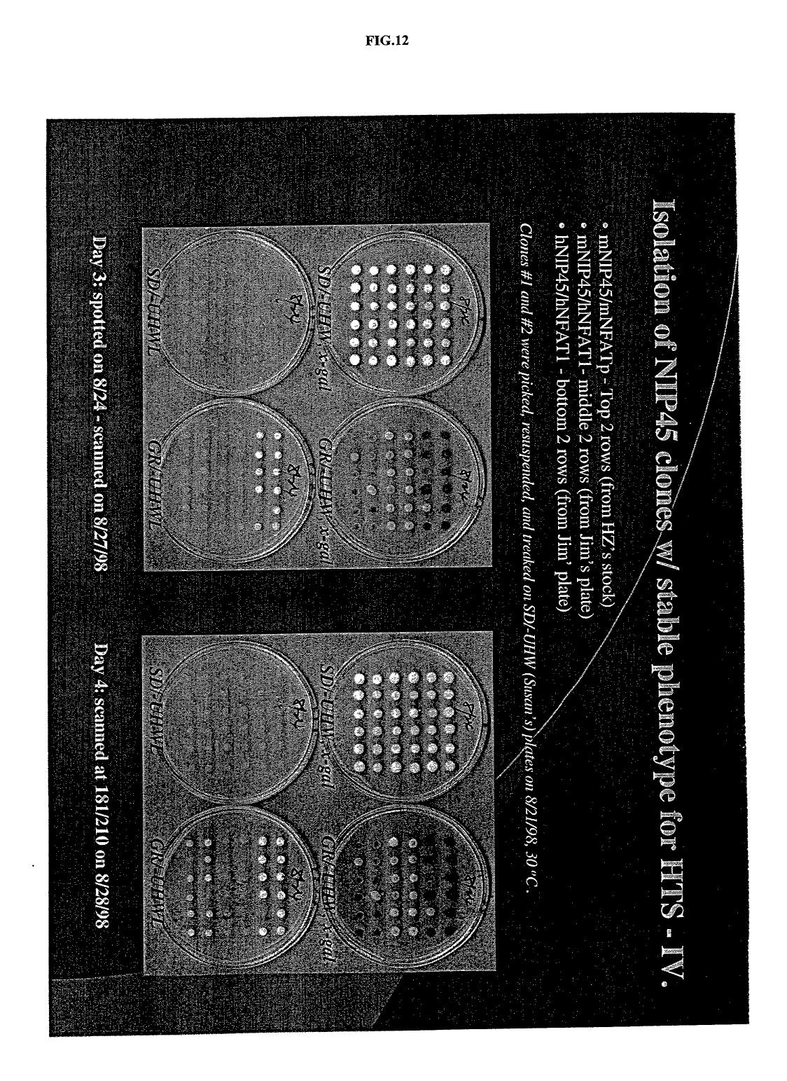

FIG. 11 and FIG. 12 show yeast two hybrid assay (described infra) results which indicate mNIP45 (murine) interacts well with mNFATp (murine) (colonies in the top two rows), hNIP45 interacts well with hNFAT1 (colonies in the bottom two rows) but slightly weaker than the interaction between mNIP45 and mNFATp, and that mNIP45 does not interact with hNFAT1 very well (colonies in the middle two rows). Hence, there is species specificity between the human and murine NIP45/NFAT interaction.

The present invention also encompasses variants of the human NIP45 trans-activator molecule SEQ ID NO:3. A preferred variant substantially as depicted in SEQ ID NO:3, for instance, is one having at least 85% amino acid sequence similarity; a more preferred variant is one having at least 90% amino acid sequence similarity; and a most preferred variant is one having at least 95% amino acid sequence similarity to the human NIP45 molecule amino acid sequence (SEQ ID NO:3) or a biologically active fragment thereof.

A “variant” of the human NIP45 molecule of the present invention may have an amino acid sequence that is different by one or more amine acid “substitutions”. The variant may have “conservative” changes, wherein a substituted amine acid has similar structural or chemical properties, eg, replacement of leucine with isoleucine. More rarely, a variant may have “nonconservative” changes, eg, replacement of a glycine with a tryptophan. Similar minor variations may also include amine acid deletions or insertions, or both. Guidance in determining which and how many amine acid residues may be substituted, inserted or deleted without abolishing biological or immunological activity may be found using computer programs well known in the art, for example, DNAStar software.

The present invention relates to nucleic acid (SEQ ID NO:1 and SEQ ID NO:2) and amino acid sequences (SEQ ID NO:3) of the novel human NIP45 and variations thereof and to the use of these sequences to identify compounds that modulate the activity of human NIP45 and the gene expression of IL-4, as described infra.

The invention further relates to the use of the human transcriptional activator molecule NIP45 in expression systems as assays for agonists or antagonists of the biomolecule. The invention also relates to the diagnosis, study, prevention, and treatment of disease related to human NIP45 and/or mediated by the transcriptional activation of IL-4.

Polynucleotide sequences which encode the human trans-activator NIP45 (SEQ ID NO:3) or a functionally equivalent derivative thereof may be used in accordance with the present invention which comprise deletions, insertions and/or substitutions of the SEQ ID NO:2 nucleic acid sequence. Biologically active variants of the human NIP45 molecule of the present invention, as well as dominant negative mutants, may also be comprised of deletions, insertions or substitutions of SEQ ID NO:3 amino acid residues. A purified polynucleotide comprising a nucleic acid sequence encoding the polypeptide having the sequence substantially as depicted in SEQ ID NO:3 or a biologically active fragment thereof is a particularly preferred embodiment of the present invention. A purified polynucleotide comprising a nucleic acid sequence which encodes a dominant negative polypeptide having the sequence substantially as depicted in SEQ ID NO:3 or a biologically-effective fragment thereof is a further embodiment of the present invention.

Amino acid substitutions of SEQ ID NO:3 may be made, for instance, on the basis of similarity in polarity, charge, solubility, hydrophobicity, hydrophilicity, and/or the amphipathic nature of the residues as long as the biological activity of the human NIP45 is retained. For example, negatively charged amino acids include aspartic acid and glutamic acid; positively charged amino acids include lysine and arginine; and amino acids with uncharged polar head groups having similar hydrophilicity values include leucine, isoleucine, valine; glycine, alanine; asparagine, glutamine; serine, threonine phenylalanine, and tyrosine.

Nucleic acid sequences which encode the amino acid sequence of the novel NIP45 transcriptional trans-activator molecule described herein are of an exponential sum due to the potential substitution of degenerate codons (different codons which encode the same amino acid). The oligonucleotide sequence selected for heterologous expression is therefore preferably tailored to meet the most common characteristic tRNA codon recognition of the particular host expression system used as well known by those skilled in the art.

Suitable conservative substitutions of amino acids are known to those of skill in this art and may be made without altering the biological activity of the resulting polypeptide, regardless of the chosen method of synthesis. The phrase “conservative substitution” includes the use of a chemically derivatized residue in place of a non-derivatized residue provided that such polypeptide displays the desired binding activity. D-isomers as well as other known derivatives may also be substituted for the naturally occurring amino acids. See, e.g., U.S. Pat. No. 5,652,369, Amino Acid Derivatives, issued Jul. 29, 1997. Substitutions, for example, may be made in accordance with those set forth in TABLE 1 as follows:

TABLE 1

| Original residue | Example substitution |
|---|---|
| Ala (A) | Gly; Ser; Val; Leu; Ile; Pro |
| Arg (R) | Lys; His; Gln; Asn |
| Asn (N) | Gln; His; Lys; Arg |
| Asp (D) | Glu |
| Cys (C) | Ser |
| Gln (Q) | Asn |
| Glu (E) | Asp |
| Gly (G) | Ala; Pro |
| His (H) | Asn; Gln; Arg; Lys |
| Ile (I) | Leu; Val; Met; Ala; Phe |
| Leu (L) | Ile; Val; Met; Ala; Phe |
| Lys (K) | Arg; Gln; His; Asn |
| Met (M) | Leu; Tyr; Ile; Phe |
| Phe (F) | Met; Leu; Tyr; Val; Ile; Ala |
| Pro (P) | Ala; Gly |
| Ser (S) | Thr |
| Thr (T) | Ser |
| Trp (W) | Tyr; Phe |
| Tyr (Y) | Trp; Phe; Thr; Ser |
| Val (V) | Ile; Leu; Met; Phe; Ala |

The nucleotide sequences of the present invention may also be engineered in order to alter a coding sequence for a variety of reasons, including but not limited to the construction of dominant negative mutant versions, alterations which modify the cloning, processing and/or expression of the gene product. For example, mutations may be introduced using techniques which are well known in the art, e.g., site-directed mutagenesis to insert new restriction sites, to alter glycosylation patterns, and the like.

Included within the scope of the present invention are alleles of the human NIP45 molecule of the present invention. As used herein, an “allele” or “allelic sequence” is an alternative form, different transcript, or splice variant of the IL-4 transcriptional trans-activator molecule described herein. Alleles result from nucleic acid mutations and mRNA splice-variants which produce polypeptides whose structure or function may or may not be altered. Any given gene may have none, one or many allelic forms. Common mutational changes which give rise to alleles are generally ascribed to natural deletions, additions or substitutions of amino acids. Each of these types of changes may occur alone, or in combination with the others, one or more times in a given sequence.

The present invention relates, in part, to the inclusion of the polynucleotide encoding the novel human NIP45 molecule in an expression vector which can be used to transform host cells or organisms. Such transgenic hosts are useful for the production of the IL-4 transcriptional trans-activator molecule and variations thereof described herein.

The nucleic acid sequence also provides for the design of antisense molecules useful in downregulating, diminishing, or eliminating expression of the genomic nucleotide sequence in cells including leukocytes, endothelial cells, and tumor or cancer cells.

The human IL4 transcriptional trans-activator molecule of the present invention (hNIP45) can also be used in screening assays to identify antagonists or inhibitors which bind, emulate substrate, or otherwise inactivate or compete with the transcription associated biomolecule. The novel NIP45 can also be used in screening assays to identify agonists which activate the transcription of IL-4 or otherwise induce the production of or prolong the lifespan of IL-4 in vivo or in vitro.

The invention also relates to pharmaceutical compounds and compositions comprising the human NIP45 molecule substantially as depicted in SEQ ID NO:3, or fragments thereof, antisense molecules capable of disrupting expression of the naturally occurring gene, and agonists, antibodies, antagonists or inhibitors of the native transcriptional activator. These compositions are useful for the prevention and/or treatment of conditions associated with abnormal expression of IL-4.

Particularly preferred embodiments of the invention are directed to methods for screening for potential immunosuppressant compounds, which interfere with or inhibit lymphokine gene activation, for example IL-4 transcriptional activation, through the hNIP45 pathway.

Overexpression of murine NIP45 in a cellular assay system using HepG2 cells has been shown to result in a 200-fold increase of endogenous IL-4 production. Hodge, M., et al., NF-AT-Driven Interleukin-4 Transcription Potentiated by NIP45, Science, 274:1903 (1996). Intervention with a compound which deprives the IL-4 promoter of the NIP45 transactivating complex is expected lead to a significant decrease of IL-4 transcription.

IL4 production by T lymphocytes and mast cells is a critical event in the development of asthma and allergic diseases. IL-4 drives T lymphocytes to the Th2 developmental pathway, leading to the production of more IL-4 and the eosinophil growth factor IL-5. IL-4 also stimulates IgE synthesis, mast cell growth, and expression of VCAM- 1 by vascular endothelial cells. These events all contribute to the pathogenesis of asthma and allergic diseases. Deprivation of hNIP45 from the IL-4 transcription complex by an antagonist agent is expected to lead to the decrease of IL-4 synthesis as has been demonstrated in cellular experimental systems. Science, 274:1903 (1996). The agent will reduce airway eosinophilia and IgE production in vivo, which is indeed relevant to controlling the principal underlying cause of asthma; chronic, eosinophilic inflammation of the airways. Moreover, the suppression of IgE synthesis by a hNIP-45 inhibitor agent will provide an opportunity for treatment of other allergic disorders. An orally active hNIP-45 inhibitor, with established efficacy in clinical trials, would have greater drug compliance and would lower health care costs. Such a compound could displace steroid therapy.

Potential diagnostic and therapeutic applications are readily apparent for modulators of the human NIP45 transcription associated biomolecule described herein. Areas which are common to disease particularly in need of therapeutic intervention include but are not limited to pathophysiological disorders manifested by dysfunctional leukocytes, T-cell activation, acute and chronic inflammatory disease, auto-immune disorders, rheumatoid arthritis, osteoarthritis, transplant rejection, macrophage regulation, endothelial cell regulation, angiogenesis, atherosclerosis, psoriasis, fibroblasts regulation, pathological fibrosis, asthma, allergic response, ARDS, atheroma, osteoarthritis, heart failure, cancer, diabetes, obesity, cachexia, Alzheimers, sepsis, neurodegeneration, and related disorders, including myasthenia gravis, Hodgkin's disease, and allergic response.

Generally Acceptable Vectors

In accordance with the present invention, polynucleotide sequences which encode the novel hNIP45, fragments of the polypeptide, fusion proteins, or functional equivalents thereof may be used in recombinant DNA molecules that direct the expression of the IL4 transcription associated biomolecule in appropriate host cells. Due to the inherent degeneracy of the genetic code, other DNA sequences which encode substantially the same or a functionally equivalent amino acid sequence, may be used to clone and express the novel trans-activator. As will be understood by those of skill in the art, it may be advantageous to produce novel hNIP45-encoding nucleotide sequences possessing non-naturally occurring codons.

Specific initiation signals may also be required for efficient translation of a hNIP45 sequence. These signals include the ATG initiation codon and adjacent sequences. In cases where the novel IL-4 transcription associated biomolecule, its initiation codon and upstream sequences are inserted into the appropriate expression vector, no additional translational control signals may be needed. However, in cases where only coding sequence, or a portion thereof, is inserted, exogenous transcriptional control signals including the ATG initiation codon must be provided. Furthermore, the initiation codon must be in the correct reading frame to ensure transcription of the entire insert. Exogenous transcriptional elements and initiation codons can be of various origins, both natural and synthetic.

Human NIP45 DNA, e.g., SEQ ID NO:2, may be recombinantly expressed to produce a biologically active IL-4 transcription associated biomolecule by molecular cloning into an expression vector containing a suitable promoter and other appropriate transcription regulatory elements, and transferred into prokaryotic or eukaryotic host cells to produce the novel polypeptide. Techniques for such manipulations are, for instance, fully described in Sambrook, J., et al., Molecular Cloning Second Edition, Cold Spring Harbor Press (1990), and are well known in the art.

Expression vectors are described herein as DNA sequences for the transcription of cloned copies of genes and the translation of their mRNAs in an appropriate host cell. Such vectors can be used to express nucleic acid sequences in a variety of hosts such as bacteria, bluegreen algae, plant cells, insect cells, fungal cells, human, and animal cells. Specifically designed vectors allow the shuttling of DNA between hosts such as bacteria-yeast, or bacteria-animal cells, or bacteria-fungal cells, or bacteria-invertebrate cells.

A variety of mammalian expression vectors may be used to express the recombinant human NIP45 molecule and variations thereof disclosed herein in mammalian cells. Commercially available mammalian expression vectors which are suitable for recombinant expression, include but are not limited to, pcDNA3 (Invitrogen), pMClneo (Stratagene), pXT1 (Stratagene), pSG5 (Stratagene), EBO-pSV2-neo (ATCC 37593) pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146), pUCTag (ATCC 37460), and 1ZD35 (ATCC 37565), pLXIN and pSIR (CLONTECH), pIRES-EGFP (CLONTECH). INVITROGEN corporation provides a wide variety of commercially available mammalian expression vector/systems which can be effectively used with the present invention. INVITROGEN, Carlsbad, Calif. See, also, PHARMINGEN products, vectors and systems, San Diego, Calif.

Baculoviral expression systems may also be used with the present invention to produce high yields of biologically active protein. Vectors such as the CLONETECH, BacPak™ Baculovirus expression system and protocols are preferred which are commercially available. CLONTECH, Palo Alto, Calif. Miller, L. K., et al., Curr. Op. Genet. Dev. 3:97 (1993); O'Reilly, D. R., et al., Baculovirus Expression Vectors: A Laboratory Manual, 127. Vectors such as the INVITROGEN, MaxBac™ Baculovirus expression system, insect cells, and protocols are also preferred which are commercially available. INVITROGEN, Carlsbad, Calif.

Example Host Cells

Host cells transformed with a nucleotide sequence which encodes a human NIP45 molecule of the present invention may be cultured under conditions suitable for the expression and recovery of the encoded protein from cell culture. Particularly preferred embodiments of the present invention are host cells transformed with a purified polynucleotide comprising a nucleic acid sequence encoding the polypeptide having the sequence substantially as depicted in SEQ ID NO:3 or a biologically active fragment thereof. Cells of this type or preparations made from them may be used to screen for pharmacologically active modulators of the IL-4 transcription associated biomolecule activity. Modulators thus identified will be used for the regulation of IL-4 transcription as defined herein.

Eukaryotic recombinant host cells are especially preferred as otherwise descibed herein or are well known to those skilled in the art. See, e.g., Ho, I-Cheng, et al., The Proto-Oncogene c-maf is Responsible for Tissue-Specific Expression of Interleukin-4, Cell, 85:973 (1996); Rao, A., et al., NFATp, A cyclosporin-sensitive transcription factor implicated in cytokine gene induction, J. Leukocyte Biology 57:536 (1995); U.S. Pat. No. 5,656,452, NF-AT[p], A T-lymphocyte DNA-Binding Protein, issued Aug. 12, 1997—and all methods therein described—are herein incorporated by reference; Marini, M. G., et al, hMaF, a small human transcription factor that heterodimerizes specifically with Nrf1 and Nrf2, J. Biol Chem Jun. 27, 1997; 272(26): 16490 (1997); Hodge et al, Science, 274:1903 (1996). Examples include but are not limited to yeast, mammalian cells including but not limited to cell lines of human, bovine, porcine, monkey and rodent origin, and insect cells including but not limited to Drosophila and silkworm derived cell lines. Cell lines derived from mammalian species which may be suitable and which are commercially available, include but are not limited to, L cells L-M(TK-) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616),BS-C-1 (ATCC CCL 26) and MRC-5 (ATCC CCL 171).

The expression vector may be introduced into host cells expressing the novel hNIP45 via any one of a number of techniques including but not limited to transformation, transfection, lipofection, protoplast fusion, and electroporation. Commercially available kits applicable for use with the present invention for hererologous expression, including well-characterized vectors, transfection reagents and conditions, and cell culture materials are well-established and readily available. CLONTECH, Palo Alto, Calif.; INVITROGEN, Carlsbad, Calif.; PHARMINGEN, San Diego, Calif.; STRATAGENE, LaJolla, Calif. The expression vector-containing cells are clonally propagated and individually analyzed to determine the level of the novel IL-4 transcription associated biomolecule production. Identification of host cell clones which express hNIP45 may be performed by several means, including but not limited to immunological reactivity with antibodies described herein, and/or the presence of host cell-associated specific hNIP45 activity, and/or the ability to covalently cross-link specific substrate to the hNIP45 with the bifunctional cross-linking reagent disuccinimidyl suberate or similar cross-linking reagents.

The transcription associated biomolecule, hNIP45, of the present invention may also be expressed as a recombinant protein with one or more additional polypeptide domains added to facilitate protein purification. Such purification facilitating domains include, but are not limited to, metal chelating peptides such as histidine-tryptophan modules that allow purification on immobilized metals (Porath, J., Protein Exp. Purif. 3:263 (1992)), protein A domains that allow purification on immobilized immunoglobulin, and the domain utilized in the FLAGS extension/affinity purification system (Immunex Corp, Seattle, Wash.). The inclusion of a cleavable linker sequences such as Factor XA or enterokinase (Invitrogen, San Diego Calif.) between the purification domain and hNIP45 is useful to facilitate purification.

Systems such as the CLONTECH, TALON™ nondenaturing protein purification kit for purifying 6xHis-tagged proteins under native conditions and protocols are preferred which are commercially available. CLONTECH, Palo Alto, Calif.

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation and acylation. Post-translational processing which cleaves a nascent form of the protein may also be important for correct insertion, folding and/or function. Different host cells such as CHO, HeLa, MDCK, 293, WI38, NIH-3T3, HEK293 etc., have specific cellular machinery and characteristic mechanisms for such post-translational activities and may be chosen to ensure the correct modification and processing of the introduced, foreign protein.

For long-term, high-yield production of recombinant proteins, stable expression is preferred. For example, cell lines which stably express the novel hNIP45 may be transformed using expression vectors which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clumps of stably transformed cells can be proliferated using tissue culture techniques appropriate to the cell type.

The human NIP45 IL-4 transcription associated biomolecule can be produced in the yeast S.cerevisiae following the insertion of the optimal cDNA cistron into expression vectors designed to direct the intracellular or extracellular expression of the heterologous protein. In the case of intracellular expression, vectors such as EmBLyex4 or the like are ligated to the beta subunit cistron. See, e.g., Rinas, U., et al., Biotechnology, 8:543 (1990); Horowitz, B., et al., J. Biol. Chem., 265:4189 (1989). For extracellular expression, a hNIP45 coding region, e.g., SEQ ID NO:2, is ligated into yeast expression vectors which may employ any of a series of well-characterized secretion signals. Levels of the expressed hNIP45 molecule are determined by the assays described herein.

A variety of protocols for detecting and measuring the expression of the novel hNIP45, using either polyclonal or monoclonal antibodies specific for the protein are known in the art. Examples include enzyme-linked immunosorbent assay (ELISA), radioimmunoassay (RIA) and fluorescent activated cell sorting (FACS). A two-site, monoclonal-based immunoassay utilizing monoclonal antibodies reactive to two non-interfering epitopes may be employed. Well known competitive binding techniques may also be employed. See, e.g., Hampton, R., et al. (1990), Serological Methods-a Laboratory Manual, APS Press, St Paul Minn.; Maddox, D. E., et al., J. Exp. Med. 158:121 1.

Various Screening Assays

The present invention is directed to methods for screening for compounds which modulate the biological activity of hNIP45 and/or the transcriptional regulation of IL-4 in vivo. Compounds which modulate these activities may be DNA, RNA, peptides, proteins, or non-proteinaceous organic molecules. Compounds may modulate the activity by increasing or attenuating the expression of DNA or RNA which encodes the human NIP45, or may antagonize or agonize the biological activity of the novel transcriptional activator itself. Compounds that modulate the expression of DNA or RNA encoding the human NIP45 or the function of the polypeptide may be detected by a variety of assays. The assay, for example a yeast two hybrid assay, may be a simple “yes/no” assay to determine whether there is a change in expression or function. The assay may be made quantitative by comparing the expression or function of a test sample with the levels of expression or function in a standard sample.

The human NIP45 described herein, its immunogenic fragments or oligopeptides can be used for screening therapeutic compounds in any of a variety of drug screening techniques. The fragment employed in such a test may be free in solution, affixed to a solid support, borne on a cell surface, or located intracellularly. The abolition of activity or the formation of binding complexes, between the human IL-4 transcription associated biomolecule and the agent being tested, may be measured. Accordingly, the present invention provides a method for screening a plurality of compounds for specific binding affinity with the human NIP45 polypeptide or a fragment thereof, comprising providing a plurality of compounds; combining the human NIP45 polypeptide of the present invention or a fragment thereof with each of a plurality of compounds for a time sufficient to allow binding under suitable conditions; and detecting binding of the trans-activator molecule, or fragment thereof, to each of the plurality of compounds, thereby identifying the compounds which specifically bind the human IL-4 transcription associated biomolecule, hNIP45.

Methods of identifying compounds that modulate the activity of a human NIP45 polypeptide are generally preferred, which comprise combining a candidate compound modulator of a human NIP45 biological activity with a polypeptide of a human NIP45 comprising the sequence substantially as depicted in SEQ ID NO:3, and measuring an effect of the candidate compound modulator on the biological activity of hNIP45 (e.g., physical interaction, transcriptional activation of the IL-4 cistron, regulation of IL-4 transcription). A further method of identifying compounds that modulate the biological activity of human NIP45, comprises combining a candidate compound modulator of human NIP45 biological activity with a host-cell expressing a NIP45 polypeptide comprising the sequence substantially as depicted in SEQ ID NO:3, and measuring an effect of the candidate compound modulator on the biological activity.

In another embodiment of the invention, a nuleic acid sequence which encodes a human NIP45 molecule substantially as depicted in SEQ ID NO:3 or a biologically active fragment thereof may be ligated to a heterologous sequence to encode a fusion protein. For example, for screening compounds for modulation of biological activity, further described infra, it may be useful to encode a chimeric hNIP45 molecule as described herein for expression in hererologous host cells. Chimeric constructs may also be used to express a ‘bait’, according to methods well known using a yeast two-hybrid system, to identify accessory native peptides that may be associated with the novel IL-4 transcription associated biomolecule described herein. Fields, S., et al., Trends Genet., 10:286 (1994); Allen, J. B., et al., TIBS, 20:511 (1995). A yeast two-hybrid system has been described wherein protein:protein interactions can be detected using a yeast-based genetic assay via reconstitution of transcriptional activators. Fields, S., Song, O., Nature 340:245 (1989). The two-hybrid system used the ability of a pair of interacting proteins to bring a transcription activation domain into close proximity with a DNA-binding site that regulates the expression of an adjacent reporter gene. Commercially available systems such as the CLONTECH, Matchmaker™ systems and protocols may be used with the present invention. CLONTECH, Palo Alto, Calif. See also, Mendelsohn, A. R., Brent, R., Curr. Op. Biotech., 5:482 (1994); Phizicky, E. M., Fields, S., Microbiological Rev., 59 (1):94 (1995); Yang, M., et al., Nucleic Acids Res., 23 (7): 1152 (1995); Fields, S., Sternglanz, R., TIG, 10 (8):286 (1994); and U.S. Pat. Nos. 5,283,173, System to Detect Protein-Protein Interactions, and 5,468,614, which are incorporated herein by reference.

A modified yeast two-hybrid system comprised of the human NIP45 homolog described herein and human NFAT1, for example, is one example embodiment to support a high throughput (HTP) screening endeavor for such a compound. Modified screening systems, for instance, can be practiced either with a positive readout or with a negative readout such as that in the recently developed versions of “Reverse Y2H” approach. See, e.g., Vidal M, Braun P. Chen E. Boeke J D, Harlow E (1996) Genetic characterization of a mammalian protein-protein interaction domain by using a yeast reverse two-hybrid system, Proc Natl Acad Sci U S A 17;93(19):10321-10326; Vidal M, Brachmann R K, Fattaey A, Harlow E, Boeke J D (1996) Reverse two-hybrid and one-hybrid systems to detect dissociation of protein-protein and DNA-protein interactions. Proc Natl Acad Sci U S A 17;93(19):10315-10320; White Mass. (1996) The yeast two-hybrid system: forward and reverse, Proc Natl Acad Sci U S A 17;93(19):10001-10003; Leanna Calif., Hannink M (1996), The reverse two-hybrid system: a genetic scheme for selection against specific protein/protein interactions, Nucleic Acids Res 1;24(17):3341-3347.

Biologically active human NIP45 and human NFAT (U.S. Pat. No. 5,656,452, NF-AT[p], A T-lymphocyte DNA-Binding Protein, issued Aug. 12, 1997) comprise sufficient components to reconstitute a yeast two-hybrid system to support HTP screening in view of the demonstrated functions, well known disease relevance, and well-defined molecular transactivation mechanism.

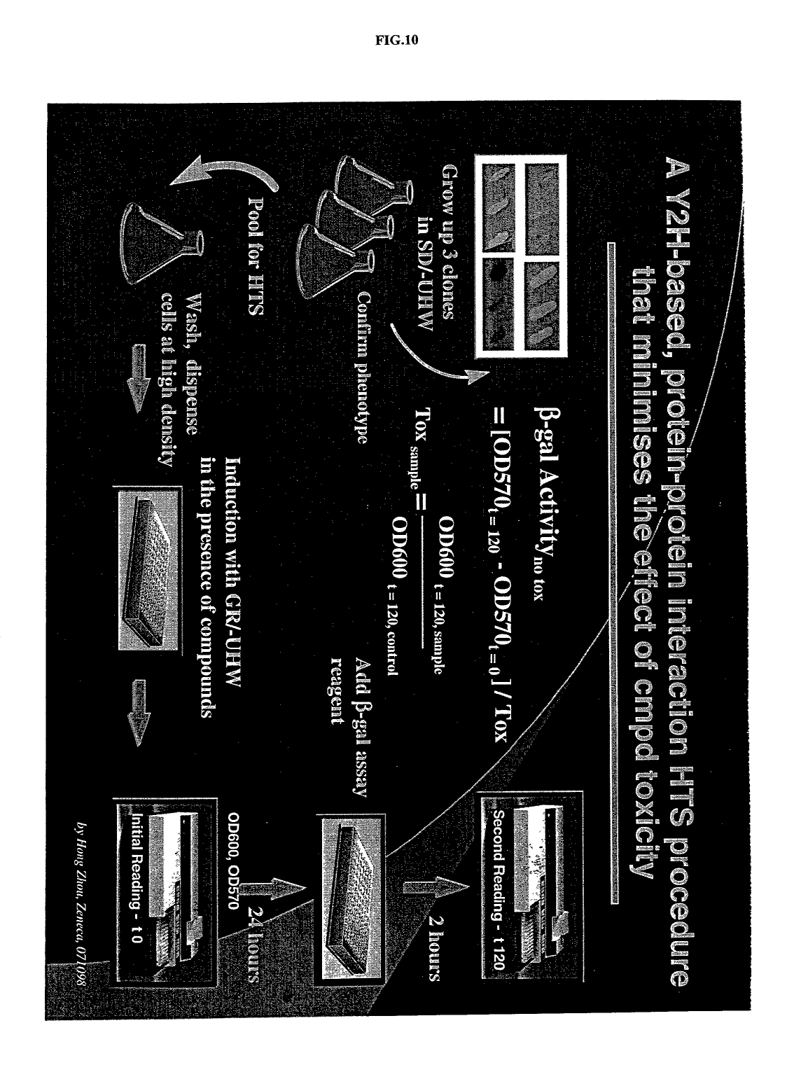

The aim of the hNIP45/hNFAT 1 high throughput screen described, for example, in Example VIII is to identify inhibitors of hNIP45/hNFAT 1 protein-protein interaction in order to block IL-4 gene activation. This assay is a LexA yeast 2-hybrid based system and was made with hNIP45 (SEQ ID NO:2) and hNFAT1 ORF cDNA. See, FIG. 9 and FIG. 10. See, also, Examples II, IV, and VI. Accordingly, methods of identifying compounds that modulate the activity of a human NIP45 polypeptide are generally preferred, which comprise combining a candidate compound modulator of a human NIP45 biological activity with a polypeptide of a human NIP45 comprising the sequence substantially as depicted in SEQ ID NO:3, and measuring an effect of the candidate compound modulator on the biological activity of hNIP45 (e.g., physical interaction). A further yeast two hybrid method of identifying compounds that modulate the biological activity of human NIP45, comprises combining a candidate compound modulator of human NIP45 biological activity with a host-cell expressing a NIP45 polypeptide comprising the sequence substantially as depicted in SEQ ID NO:3, and measuring an effect of the candidate compound modulator on the biological activity.

The particular procedure described in Example VIII was specifically developed in order to obtain high throughput screening hits specific for hNIP45. In previous yeast 2-hybrid (Y2H) based HTS, the LEU2 reporter have been used for readout. Assays which use the LEU2 reporter, readout data is growth/no growth. Because the host yeast is defective in generating leucine, leucine-independent growth is dependent on the reporter activation, which is dependent on an effective physical interaction between the bait and prey fusions. If such an interaction is blocked or disrupted by a compound, the cells will not be able to grow in the absence of leucine, hence, a phenotype of “no growth”. The completeness of such blockade is determined by the specificity and affinity of such a compound with the target(s). However, one can easily perceive that a compound toxic to the yeast cell will also result in a “no growth” readout phenotype and that compound will be end up in the resulting “hits” of such HTS. The toxicity of the compounds vary. The much higher hit rate resulting from Y2H-based HTS which uses “grow/no growth” as a simply readout make it significantly difficult for the scientists to decide which one to pursue further. As the lead identification (LI) is a complicated multistep process, this high false positive rate likely to hide the real interesting hits away from an immediate testing. Therefore, a procedure that will make the HTS hits more specific is extremely important in the highly competitive modern drug R&D. A modified version of the conventional yeast 2-hybrid is set forth herein which will exclude the effect of compound toxicity. This modified version of Y2H is termed “reverse yeast 2-hybrid”. In this system, a reporter gene that is toxic to the cell is used. Upon the interaction between the bait and prey, the reporter is activated, the toxic gene is expressed and the host cell is killed. A compound that blocks the interaction, however, will lead the “growth” of the test cells. Therefore, those can not block the interaction and those toxic to the cells by them selves will be dropped from the readout.

Example VIII provides an example protocol wherein cells are grown to high density under suppressive condition (SD/-UHW), washed (to remove the suppressor, glucose), resuspended in high density (OD600=1.0) and induced for expression with GR/-UIIW in the presence of test compounds. Because the cells have been plated at high density, the growth potential of the cells are limited. Thus, the effect of the compound toxicity on-cell density will be minimal. The resulting amount of b-gal will be dependent on the efficacy of the compound to block the bait-prey interaction. To developed the result, b-gal reagent is added to the test plates on the next day and initial OD600 (for cell density) and OD570 (for b-gal product) measured. After two hour incubation at 30° C., the end OD600 and OD570 are taken. After calculation using the provided equations, inhibition efficiency of each compound is assessed resulting from blockage of the bait-prey interaction, with minimal effect from compound toxicity.

An “IL-4 cistron” refers to an IL-4 promoter sequence and reporter gene, which in one preferred embodiment is the IL-4 structural coding region, as well as transcription associated biomolecules required for expression of the reporter gene. “Transcription associated biomolecules” refer to factors that are directly or indirectly associated with transcriptional regulation including but not limited to transcriptional activators NFAT (NFATp and/or NFAT1), c-MAF, and NIP45. See, e.g., FIG. 7. Regulation of transcription refers to down regulation via antagonization, repression, neutralization, or sequestration, of a transcription associated biomolecule including but not limited to NIP45; as well as up regulation via transcriptional activation including but not limited to the biological activity of a NIP45 molecule described herein or agonization thereof by a compound identified by means described herein; as well as up regulation via antagonisation, neutralization, or sequestration of a repressor. See, Examples II and III. Accordingly, another preferred method is one of identifying compounds that modulate the transcriptional activation of IL-4, comprising combining a candidate compound modulator of transcriptional activation of IL-4 with a polypeptide of a human trans-activator having the sequence substantially as depicted in SEQ ID NO:3, and an IL-4 cistron, and measuring an effect of the candidate compound modulator on the transcriptional activation of the IL-4 cistron.

Methods of identifying compounds that modulate the activity of an hNIP45 or modulate or regulate IL-4 transcription, are also preferred which comprise combining a candidate compound modulator of transcriptional activation of IL-4 with a host-cell comprising an IL-4 cistron and expressing (or capable of expressing hNIP45 via e.g., inducible expression) the polypeptide of a human NIP45 molecule having the sequence substantially as depicted in SEQ ID NO:3, and measuring an effect of the candidate compound modulator on the transcriptional activation of the IL-4 cistron. Preferred cellular assays of for modulators fall into two general categories: 1) direct measurement of the physical hNIP45 biological activity, and 2) measurement of transcriptional activation of the IL-4 cistron. These methods can employ the endogenous hNIP45, or overexpressed recombinant hNIP45.

In order to measure the cellular activity of the transcriptional activator, the source may be a whole cell lysate, prepared by one to three freeze-thaw cycles in the presence of standard protease inhibitors. Alternatively, the hNIP45 may be partially or completely purified by standard protein purification methods. Finally, the hNIP45 may be purified by affinity chromatography using specific antibody described herein or by ligands specific for an epitope tag engineered into the recombinant molecule moreover described herein. The preparation may then be assayed for activity as described.

Compounds which are identified generally according to methods descibed, referenced, and contemplated herein that modulate the activity of human NIP45 (most preferably regulate IL-4 transcription) are especially preferred embodiments of the present invention.

Purified polypeptides comprising the amino acid sequence substantially as depicted in SEQ ID NO:3 are especially preferred embodiments of the present invention.

An especially preferred embodiment of the present invention is a method for treatment of a patient in need of such treatment for a condition which is mediated by the human NIP45 described herein comprising administration of a therapeutically effective amount of a human NIP45 modulating compound.

Another especially preferred embodiment of the present invention is a method for treatment of a patient in need of such treatment for a condition which is mediated by transcriptional activation of IL-4 comprising administration of a therapeutically effective amount of a human NIP45 modulating compound.

A further especially preferred embodiment of the present invention is a method for treatment of a patient in need of such treatment for a condition which is mediated by transcriptional activation of IL-4 comprising administration of a therapeutically effective amount of a compound modulator of transcriptional activation of IL-4.

Antibodies

Monospecific antibodies to the IL-4 transcription associated biomolecule, hNIP45, of the present invention are purified from mammalian antisera containing antibodies reactive against the polypeptide or are prepared as monoclonal antibodies reactive with an hNIP45 polypeptide using the technique of Kohler and Milstein, Nature, 256:495 (1975). Mono-specific antibody as used herein is defined as a single antibody species or multiple antibody species with homogenous binding characteristics for the novel hNIP45. Homogenous binding as used herein refers to the ability of the antibody species to bind to a specific antigen or epitope, such as those associated with the novel transcription activator, as described. Human NIP45 specific antibodies are raised by immunizing animals such as mice, rats, guinea pigs, rabbits, goats, horses and the like, with rabbits being preferred, with an appropriate concentration of the human NIP45 either with or without an immune adjuvant.

Preimmune serum is collected prior to the first immunization. Each animal receives between about 0.1 mg and about 1000 mg of hNIP45 polypeptide associated with an acceptable immune adjuvant. Such acceptable adjuvants include, but are not limited to, Freund's complete, Freund's incomplete, alum-precipitate, water in oil emulsion containing Corynebacterium parvum and tRNA. The initial immunization consists of a hNIP45 polypeptide in, preferably, Freund's complete adjuvant at multiple sites either subcutaneously (SC), intraperitoneally (IP) or both. Each animal is bled at regular intervals, preferably weekly, to determine antibody titer. The animals may or may not receive booster injections following the initial immunization. Those animals receiving booster injections are generally given an equal amount of the antigen in Freund's incomplete adjuvant by the same route. Booster injections are given at about three week intervals until maximal titers are obtained. At about 7 days after each booster immunization or about weekly after a single immunization, the animals are bled, the serum collected, and aliquots are stored at about −20°C.

Monoclonal antibodies (mAb) reactive with the hNIP45 polypeptide are prepared by immunizing inbred mice, preferably Balb/c, with a hNIP45 polypeptide. The mice are immunized by the IP or SC route with about 0.1 mg to about 10 mg, preferably about 1 mg, of hNIP45 polypeptide in about 0.5 ml buffer or saline incorporated in an equal volume of an acceptable adjuvant, as discussed above. Freund's complete adjuvant is preferred. The mice receive an initial immunization on day 0 and are rested for about 3 to about 30 weeks. Immunized mice are given one or more booster immunizations of about 0.1 to about 10 mg of hNIP45 polypeptide in a buffer solution such as phosphate buffered saline by the intravenous (IV) route. Lymphocytes, from antibody positive mice, preferably splenic lymphocytes, are obtained by removing spleens from immunized mice by standard procedures known in the art. Hybridoma cells are produced by mixing the splenic lymphocytes with an appropriate fusion partner, preferably myeloma cells, under conditions which will allow the formation of stable hybridomas. Fusion partners may include, but are not limited to: mouse myelomas P3/NS 1/Ag 4-1; MPC-11; S-194 and Sp 2/0, with Sp 2/0 being preferred. The antibody producing cells and myeloma cells are fused in polyethylene glycol, about 1000 molecular weight, at concentrations from about 30% to about 50%. Fused hybridoma cells are selected by growth in hypoxanthine, thymidine and aminopterin supplemented Dulbecco's Modified Eagles Medium (DMEM) by procedures known in the art. Supernatant fluids are collected from growth positive wells on about days 14, 18, and 21 and are screened for antibody production by an immunoassay such as solid phase immunoradioassay (SPIRA) using the human NIP45 polypeptide as the antigen. The culture fluids are also tested in the Ouchterlony precipitation assay to determine the isotype of the mAb. Hybridoma cells from antibody positive wells are cloned by a technique such as the soft agar technique of MacPherson, Soft Agar Techniques, in Tissue Culture Methods and Applications, Kruse and Paterson, Eds., Academic Press, 1973.

Monoclonal antibodies are produced in vivo by injection of pristane primed Balb/c mice, approximately 0.5 ml per mouse, with about 2×10⁶ to about 6×10⁶ hybridoma cells about 4 days after priming. Ascites fluid is collected at approximately 8-12 days after cell transfer and the monoclonal antibodies are purified by techniques known in the art.

In vitro production of the anti- human NIP45 polypeptide mAb is carried out by growing the hydridoma in DMEM containing about 2% fetal calf serum to obtain sufficient quantities of the specific mAb. The mAb are purified by techniques known in the art.

Diagnostic Assays

Antibody titers of ascites or hybridoma culture fluids are determined by various serological or immunological assays which include, but are not limited to, precipitation, passive agglutination, enzyme-linked immunosorbent antibody (ELISA) technique and radioimmunoassay (RIA) techniques. Similar diagnostic assays are used to detect the presence of the novel IL-4 transcription associated biomolecule in body fluids or tissue and cell extracts.

Diagnostic assays using the human NIP45 polypeptide specific antibodies are useful for the diagnosis of conditions, disorders or diseases characterized by abnormal expression of hNIP45 or expression of genes associated with abnormal cell growth. Diagnostic assays for the IL-4 transcription associated biomolecule of this invention include methods utilizing the antibody and a label to detect the human NIP45 polypeptide in human body fluids, cells, tissues or sections or extracts of such tissues. The polypeptides and antibodies of the present invention may be used with or without modification. Frequently, the polypeptides and antibodies will be labeled by joining them, either covalently or noncovalently, with a reporter molecule, a myriad of which are well-known to those skilled in the art.

A variety of protocols for measuring the hNIP45 polypeptide, using either polyclonal or monoclonal antibodies specific for the respective protein are known in the art. Examples include enzyme-linked immunosorbent assay (ELISA), radioimmunoassay (RIA) and fluorescent activated cell sorting (FACS). A two-site, monoclonal-based immunoassay utilizing monoclonal antibodies reactive to two non-interfering epitopes on the human NIP45 polypeptide is preferred, but a competitive binding assay may be employed. These assays are described, among other places, in Maddox, D. E. et al., J. Exp. Med. 158:1211 (1983); Sites, D. P., et al., Basic and Clinical Immunology, Ch.22, 4th Ed., Lange Medical Publications, Los Altos, Calif. (1982); U.S. Pat. Nos. 3,654,090, 3,850,752; and 4,016,043.

In order to provide a basis for the diagnosis of disease, normal or standard values for the human NIP45 polypeptide expression must be established. This is accomplished by combining body fluids or cell extracts taken from normal subjects, either animal or human, with antibody to the human IL-4 transcription associated biomolecule under conditions suitable for complex formation which are well known in the art. The amount of standard complex formation may be quantified by comparing it with a dilution series of positive controls where a known amount of antibody is combined with known concentrations of purified human NIP45 polypeptide. Then, standard values obtained from normal samples may be compared with values obtained from samples from subjects potentially affected by a disorder or disease related to the human IL-4 transcription associated biomolecule expression. Deviation between standard and subject values establishes the presence of the disease state.

Kits containing human NIP45 nucleic acid, antibodies to a hNIP45 polpeptide, or protein may be prepared. Such kits are used to detect heterologous nucleic acid which hybridizes to hNIP45 nucleic acid, or to detect the presence of protein or peptide fragments in a sample. Such characterization is useful for a variety of purposes including, but not limited to, forensic analyses and epidemiological studies.

The DNA molecules, RNA molecules, recombinant protein and antibodies of the present invention may be used to screen and measure levels of the novel hNIP45 DNA, RNA or protein. The recombinant proteins, DNA molecules, RNA molecules and antibodies lend themselves to the formulation of kits suitable for the detection and typing of the novel human IL-4 transcription associated biomolecule. Such a kit would comprise a compartmentalized carrier suitable to hold in close confinement at least one container. The carrier would further comprise reagents such as recombinant human NIP45 or anti-hNIP45 antibodies suitable for detecting the novel IL-4 transcription associated biomolecule. The carrier may also contain a means for detection such as labeled antigen or enzyme substrates or the like.

Polynucleotide sequences which encode the novel hNIP45 may be used for the diagnosis of conditions or diseases with which the expression of the novel IL-4 transcription associated biomolecule is associated. For example, polynucleotide sequences encoding hNIP45 may be used in hybridization or PCR assays of fluids or tissues from biopsies to detect expression of the IL-4 trans-activator. The form of such qualitative or quantitative methods may include Southern or northern analysis, dot blot or other membrane-based technologies; PCR technologies; dip stick, pin, chip and ELISA technologies. All of these techniques are well known in the art and are the basis of many commercially available diagnostic kits. Such assays may also be used to evaluate the efficacy of a particular therapeutic treatment regime in animal studies, in clinical trials, or in monitoring the treatment of an individual patient. Once disease is established, a therapeutic agent is administered and a treatment profile is generated. Such assays may be repeated on a regular basis to evaluate whether the values in the profile progress toward or return to the normal or standard pattern. Successive treatment profiles may be used to show the efficacy of treatment over a period of several days or several months.

Polynucleotide sequences which encode the novel human IL-4 transcription activator may also be employed in analyses to map chromosomal locations, e.g., screening for functional association with disease markers. Moreover the sequences described herein are contemplated for use to identify human sequence polymorphisms and possible association with disease as well as analyses to select optimal sequence from among possible polymorphic sequences for the design of compounds to modulate the hNIP45 biological activity and therefore regulate IL-4 transcription, most preferably in vivo. Furthermore the sequences are contemplated as screening tools for use in the identification of appropriate human subjects and patients for therapeutic clinical trials.

Purification Via Affinity Columns

It is readily apparent to those skilled in the art that methods for producing antibodies may be utilized to produce antibodies specific for hNIP45 polypeptide fragments, or the full-length nascent human NIP45 polypeptide. Specifically, it is readily apparent to those skilled in the art that antibodies may be generated which are specific for the fully functional receptor or fragments thereof.

Human NIP45 polypeptide antibody affinity columns are made by adding the antibodies to Affigel-10 (Biorad), a gel support which is activated with N hydroxysuccinimide esters such that the antibodies form covalent linkages with the agarose gel bead support. The antibodies are then coupled to the gel via amide bonds with the spacer arm. The remaining activated esters are then quenched with 1M ethanolamine HCl (pH 8). The column is washed with water followed by 0.23M glycine HCl (pH 2.6) to remove any non-conjugated antibody or extraneous protein. The column is then equilibrated in phosphate buffered saline (pH 7.3) with appropriate detergent and the cell culture supernatants or cell extracts containing human NIP45 polypeptide made using appropriate membrane solubilizing detergents are slowly passed through the column. The column is then washed with phosphate buffered saline/detergent until the optical density falls to background, then the protein is eluted with 0.23M glycine-HCl (pH 2.6)/detergent. The purified human NIP45 polypeptide is then dialyzed against phosphate buffered saline/detergent.

Recombinant hNIP45 molecules can be separated from other cellular proteins by use of an immunoaffinity column made with monoclonal or polyclonal antibodies specific for full length nascent human NIP45 polypeptide, or polypeptide fragments of the hNIP45 molecule. Human NIP45 polypeptides described herein may be used to affinity purify biological effectors from native biological materials, e.g. disease tissue. Affinity chromatography techniques are well known to those skilled in the art. A human NIP45 peptide described herein or an effective fragment thereof, is fixed to a solid matrix, e.g. CNBr activated Sepharose according to the protocol of the supplier (Pharmacia, Piscataway, N.J.), and a homogenized/buffered cellular solution containing a potential molecule of interest is passed through the column. After washing, the column retains only the biological effector which is subsequently eluted, e.g., using 0.5M acetic acid or a NaCl gradient.

Antisense Molecules

The cDNA sequence SEQ ID NO:1 provided herein, may be used in another embodiment of the invention to study the physiological relevance of the novel human NIP45 in cells, especially cells of hematopoietic origin, by knocking out the endogenous gene by use of anti-sense constructs.

To enable methods of down-regulating expression of the hNIP45 of the present invention in mammalian cells, an example antisense expression construct containing the complement DNA sequence to the sequence substantially as depicted in SEQ ID NO:2 can be readily constructed for instance using the pREP 10 vector (Invitrogen Corporation). Transcripts are expected to inhibit translation of the wild-type hNIP45 mRNA in cells transfected with this type construct. Transcript are, in principle, effective for inhibiting translation of the transcript, and capable of inducing the effects (e.g., regulation of IL-4 transcription) herein described. Translation is most effectively inhibited by blocking the mRNA at a site at or near the initiation codon. Thus, oligonucleotides complementary to the corresponding 5 ′-terminal region of the human NIP45 mRNA transcript are preferred. Secondary or tertiary structure which might interfere with hybridization is minimal in this region. Moreover, sequences that are too distant in the 3′ direction from the initiation site can be less effective in hybridizing the mRNA transcripts because of a “read-through” phenomenon whereby the ribosome appears to unravel the antisense/sense duplex to permit translation of the message. Oligonucleotides which are complementary to and hybridizable with any portion of the novel human NIP45 mRNA are contemplated for therapeutic use. U.S. Pat. No. 5,639,595, Identification of Novel Drugs and Reagents, issued Jun. 17, 1997, wherein methods of identifying oligonucleotide sequences that display in vivo activity are thoroughly described, is herein incorporated by reference. Expression vectors containing random oligonucleotide sequences derived from previously known polynucleotides are transformed into cells. The cells are then assayed for a phenotype resulting from the desired activity of the oligonucleotide. Once cells with the desired phenotype have been identified, the sequence of the oligonucleotide having the desired activity can be identified. Identification may be accomplished by recovering the vector or by polymerase chain reaction (PCR) amplification and sequencing the region containing the inserted nucleic acid material.

Nucleotide sequences that are complementary to the novel hNIP45 polypeptide encoding polynucleotide sequence can be synthesized for antisense therapy. These antisense molecules may be DNA, stable derivatives of DNA such as phosphorothioates or methylphosphonates, RNA, stable derivatives of RNA such as 2′-O-alkylRNA, or other oligonucleotide mimetics. U.S. Pat. No. 5,652,355, Hybrid Oligonucleotide Phosphorothioates, issued Jul. 29, 1997, and U.S. Pat. No. 5,652,356, Inverted Chimeric and Hybrid Oligonucleotides, issued Jul. 29, 1997, which describe the synthesis and effect of physiologically-stable antisense molecules, are incorporated by reference. Human NIP45 antisense molecules may be introduced into cells by microinjection, liposome encapsulation or by expression from vectors harboring the antisense sequence. Antisense therapy may be particularly useful for the treatment of diseases where it is beneficial to modulate IL-4 gene expression.

Gene Therapy

A human NIP45 polypeptide described herein may administered to a subject via gene therapy. A polypeptide of the present invention may be delivered to the cells of target organs in this manner. Conversely, hNIP45 polypeptide antisense gene therapy may be used to modulate the expression of the polypeptide in the cells of target organs and hence regulate IL-4 transcription. The human NIP45 polypeptide coding region can be ligated into viral vectors which mediate transfer of the trans-activator polypeptide nucleic acid by infection of recipient host cells. Suitable viral vectors include retrovirus, adenovirus, adeno-associated virus, herpes virus, vaccinia virus, polio virus and the like. See, e.g., U.S. Pat. No. 5,624,820, Episomal Expression Vector for Human Gene Therapy, issued Apr. 29, 1997. Nucleic acid coding regions of the present invention are incorporated into effective eukaryotic expression vectors, which are directly administered or introduced into somatic cells for gene therapy (a nucleic acid fragment comprising a coding region, preferably mRNA transcripts, may also be administered directly or introduced into somatic cells). See, e.g., U.S. Pat. No. 5,589,466, issued Dec. 31, 1996. Such nucleic acids and vectors may remain episomal or may be incorporated into the host chromosomal DNA as a provirus or portion thereof that includes the gene fusion and appropriate eukaryotic transcription and translation signals, i.e, an effectively positioned RNA polymerase promoter 5′ to the transcriptional start site and ATG translation initiation codon of the gene fusion as well as termination codon(s) and transcript polyadenylation signals effectively positioned 3′ to the coding region. Alternatively, the human NIP45 polypeptide DNA can be transferred into cells for gene therapy by non-viral techniques including receptor-mediated targeted DNA transfer using ligand-DNA conjugates or adenovirus-ligand-DNA conjugates, lipofection membrane fusion or direct microinjection. These procedures and variations thereof are suitable for ex vivo, as well as in vivo human NIP45 gene therapy according to established methods in this art.

PCR Diagnostics

The nucleic acid sequence, oligonucleotides, fragments, portions or antisense molecules thereof, may be used in diagnostic assays of body fluids or biopsied tissues to detect the expression level of the novel human NIP45 molecule. For example, sequences designed from the cDNA sequence SEQ ID NO:1 or sequences comprised in SEQ ID NO:2 can be used to detect the presence of the mRNA transcripts in a patient or to monitor the modulation of transcripts during treatment.

One method for amplification of target nucleic acids, or for later analysis by hybridization assays, is known as the polymerase chain reaction (“PCR”) or PCR technique. The PCR technique can be applied to detect sequences of the invention in suspected samples using oligonucleotide primers spaced apart from each other and based on the genetic sequence, e.g., SEQ ID NO:1, set forth herein. The primers are complementary to opposite strands of a double stranded DNA molecule and are typically separated by from about 50 to 450 nucleotides or more (usually not more than 2000 nucleotides). This method entails preparing the specific oligonucleotide primers followed by repeated cycles of target DNA denaturation, primer binding, and extension with a DNA polymerase to obtain DNA fragments of the expected length based on the primer spacing. One example embodiment of the present invention is a diagnostic composition for the identification of a polynucleotide sequence comprising the sequence substantially as depicted in SEQ ID NO:2 comprising PCR primers derived from SEQ ID NO:1. The degree of amplification of a target sequence is controlled by the number of cycles that are performed and is theoretically calculated by the simple formula 2n where n is the number of cycles. See, e.g., Perkin Elmer, PCR Bibliography, Roche Molecular Systems, Branchburg, N.J.; CLONTECH products, Palo Alto, Calif.; U.S. Pat. No. 5,629,158, Solid Phase Diagnosis of Medical Conditions, issued May 13, 1997.

Compositions

Pharmaceutically useful compositions comprising sequences pertaing to the novel human NIP45 polypeptide DNA, RNA, antisense sequences, or the human oNIP45 polypeptide itself, or variants and analogs which have the human NIP45 biological activity or otherwise modulate IL-4 transcription, may be formulated according to known methods such as by the admixture of a pharmaceutically acceptable carrier. Examples of such carriers and methods of formulation may be found in Remington's Pharmaceutical Sciences (Maack Publishing Co, Easton, Penn.). To form a pharmaceutically acceptable composition suitable for effective administration, such compositions will contain an effective amount of the protein, DNA, RNA, or compound modulator.

Therapeutic or diagnostic compositions of the invention are administered to an individual in amounts sufficient to treat or diagnose human NIP45 related disorders or IL-4 gene expression related disorders. The effective amount may vary according to a variety of factors such as the individual's condition, weight, sex and age. Other factors include the mode of administration.

The term “chemical derivative” describes a molecule that contains additional chemical moieties which are not normally a part of the base molecule. Such moieties may improve the solubility, half-life, absorption, etc. of the base molecule. Alternatively the moieties may attenuate undesirable side effects of the base molecule or decrease the toxicity of the base molecule. Examples of such moieties are described in a variety of texts, such as Remington's Pharmaceutical Sciences.

Pharmaceutical compositions suitable for use in the present invention include compositions wherein the active ingredients are contained in an effective amount to achieve the intended purpose. The determination of an effective dose is well within the -capability of those skilled in the art. The therapeutically effective dose can be estimated initially either in cell culture assays, eg, of neoplastic cells, or in animal models, usually mice, rabbits, dogs, or pigs. The animal model is also used to achieve a desirable concentration range and route of administration. Such information can then be used to determine useful doses and routes for administration in humans. A therapeutically effective dose refers to that amount of protein or its antibodies, antagonists, or inhibitors which ameliorate the symptoms or condition. The exact dosage is chosen by the individual physician in view of the patient to be treated.

Compounds identified according to the methods disclosed herein may be used alone at appropriate dosages defined by routine testing in order to obtain optimal modulation of hNIP45 biological activity and/or IL-4 gene expression, or its activity while minimizing any potential toxicity. In addition, co-administration or sequential administration of other agents may be desirable.

The pharmaceutical compositions may be provided to the individual by a variety of routes such as subcutaneous, topical, oral and intramuscular. Administration of pharmaceutical compositions is accomplished orally or parenterally. Methods of parenteral delivery include topical, intra-arterial (directly to the tissue), intramuscular, subcutaneous, intramedullary, intrathecal, intraventricular, intravenous, intraperitoneal, or intranasal administration. The present invention also has the objective of providing suitable topical, oral, systemic and parenteral pharmaceutical formulations for use in the novel methods of treatment of the present invention. The compositions containing compounds identified according to this invention as the active ingredient for use in the modulation of hNIP45 can be administered in a wide variety of therapeutic dosage forms in conventional vehicles for administration. For example, the compounds can be administered in such oral dosage forms as tablets, capsules (each including timed release and sustained release formulations), pills, powders, granules, elixirs, tinctures, solutions, suspensions, syrups and emulsions, or by injection. Likewise, they may also be administered in intravenous (both bolus and infusion), intraperitoneal, subcutaneous, topical with or without occlusion, or intramuscular form, all using forms well known to those of ordinary skill in the pharmaceutical arts. An effective but non-toxic amount of the compound desired can be employed as a hNIP45 modulating agent. The daily dosage of the products may be varied over a wide range from 0.01 to 1,000 mg per adult human/per day. For oral administration, the compositions are preferably provided in the form of scored or unscored tablets containing 0.01, 0.05, 0.1, 0.5, 1.0, 2.5, 5.0, 10.0, 15.0, 25.0, and 50.0 milligrams of the active ingredient for the symptomatic adjustment of the dosage to the patient to be treated. An effective amount of the drug is ordinarily supplied at a dosage level of from about 0.0001 mg/kg to about 100 mg/kg of body weight per day. The range is more particularly from about 0.001 mg/kg to 10 mg/kg of body weight per day. Even more particularly, the range varies from about 0.05 to about 1 mg/kg. Of course the dosage level will vary depending upon the potency of the particular compound. Certain compounds will be more potent than others. In addition, the dosage level will vary depending upon the bioavailability of the compound. The more bioavailable and potent the compound, the less compound will need to be administered through any delivery route, including but not limited to oral delivery. The dosages of the human NIP45 modulators are adjusted when combined to achieve desired effects. On the other hand, dosages of these various agents may be independently optimized and combined to achieve a synergistic result wherein the pathology is reduced more than it would be if either agent were used alone. Those skilled in the art will employ different formulations for nucleotides than for proteins or their inhibitors. Similarly, delivery of polynucleotides or polypeptides will be specific to particular cells and conditions.

### EXAMPLES

### Example I

The human homolog (hNIP45) of IL-4 gene transactivator, NFATp-interacting protein (NIP45), (cDNA described herein as SEQ ID NO:1) was isolated from a human lymph node cDNA library using probes prepared from murine NIP45 (mNIP45) cDNA. A gt10 lambda phage library made of human lymph node cDNA (CLONTECH, cat #:HL5000a) was used to isolate the human homolog of murine NIP45.

Probe Preparation for Library Screening

DNA probes were prepared from the mNIP45 cDNA. Hodge, M., et al., NF-AT-Driven Interleukin-4 Transcription Potentiated by NIP 45, Science, 274:1903 (1996). The full length cDNA sequence is also available in GeneBank [U76759] which can be used to design primers to generate cDNA fragments by PCR and further to prepare probes using the same method. To prepare the probes for library screening, a plasmid containing the sequence was first digested with restriction enzyme, Sal I; a fragment of 3 Kb which contains the mouse NIP45 cDNA was gel-purified and the probes were prepared from this cDNA fragment by using a commercial random labeling kit (Pharmacia, cat.#27-9240-01).

Library Screening

Approximately 75,000 bacteriophage plaques of the human lymph node cDNA library were plated and screened with the prepared radioactive probes. Two sets of nylon membrane lifts were made from these plates and pre-hybridized with Solution 1 (50% formamide, 5×SSPE, 5×Denhardt's solution, 0.1% SDS and 100 mg/ml denatured salmon sperm DNA) at 42° C. for 4 hours. The membrane lifts were then hybridized with the prepared NIP45 probes in the same solution at 42° C. overnight. After hybridization, the lifts were washed once with wash buffer 1 (2×SSC, 0.5% SDS) at room temperature for 15 minutes, and twice with wash buffer 2 (1×SSC, 0.1% SDS), each at 65° C. for 60 minutes—then wrapped and exposed to X-Ray films at −80° C. for 4 days. Positive spots on both duplicate membrane lifts were considered as primary candidates. Phage plaques corresponding to the candidates were isolated and plated and re-screened with the freshly prepared probes using a ECL kit from Amersham (Amersham Cat# RPN3000). After two rounds of screening with the ECL kit, 10 positive candidate clones were identified.

Restriction Analysis

To reveal the cDNA sequence identity of the 10 positive candidate phage clones, the phage DNA were prepared and digested with EcoRI. The size of the digests were assessed by agarose gel electrophoresis and their homology with the NIP45 sequence were confirmed by southern analysis using the same probes. The cDNA fragments released by EcoRI digestion were further subcloned into the EcoRI site of pCI vector (Promega, cat# E1731). The resulting pCI clones were subjected to sequencing analysis.

Sequencing Analysis

The initial sequencing analyses were performed with T7 forward and reverse universal primers with the automated sequencer (ABI 371). Further sequencing were carried out with gene-specific primers designed based on resulting sequences. Sequencing results revealed that two clones representative of the novel hNIP45 had inserts of 2561 bp and 3087 bp in length, respectively. Further analysis indicated that the two clones were representative of one contiguous sequence. The overlapped part of the these two sequences were identical.

### EXAMPLE II

Functional Characterization

The hNIP45 as described herein may be functionally characterized, for instance, according to the following examples:

1. In yeast for hNIP45 interaction with NFATp to activate reporter genes.

An hNIP45 AD-fusion construct is co-transformed into the yeast host EGY48 (CLONTECH, cat # K1609-1) with the original murine LexA-NFATp bait and reporter, pSH18-34 (CLONTECH, cat# K1609-1). The functional conservation of hNIP45 is assessed by its ability of interacting with the NFATp protein to activate the reporter gene expression. Hodge, M., et al., NF-AT-Driven Interleukin-4 Transcription Potentiated by NIP45, Science, 274:1903 (1996). See, Example VI infra.

2. Mammalian cells analyses, e.g., Jurkat and HepG2, etc., for modulation of IL-4 gene expression.

The proximal promoter which controls tissue-specific expression of the human IL-4 gene may be used (FIG. 6). The proximal promoter of the IL-2 gene may be be similarly used for control purpose. The effect of hNIP45 on IL-4 promoter is assessed in Jurkat cells as well as HepG2, etc. Hodge, M., et al., NF-AT-Driven Interleukin-4 Transcription Potentiated by NIP45, Science, 274:1903 (1996).

### EXAMPLE III

IL-4 promoter based cellular high throughput screening systems

1. Dephosphorylation of NFATp

by stimulation with ionomycin and phorbol 12-myristate 13-acetate;

by coexpression of constitutively active calcineurin;

by over-expression of NFATp (or NFAT1).

2. Presence of c-maf, by Ectopic expression of c-maf and possibly

by pre-treating the cells with IL-4 (e.g. treat B cells with IL-4 and then remove).

3. Presence of the novel NIP45 human homolog described herein.

Jurkat cells, for example, are convenient host cells for constructing the cellular HTP system to examine IL-4 promoter transactivation. See, e.g., Klein-Hessling S. Schneider G. Heinfling A. Chuvpilo S. Serfling E. (1993) HMG I(Y) interferes with the DNA binding of NF-AT factors and the induction of the interleukin 4 promoter in T cells. Proceedings of the National Academy of Sciences of the United States of America. 93 (26):15311-6, 1996; Paliogianni F. Boumpas D T. (1996) Prostaglandin E2 inhibits the nuclear transcription of the human interleukin 2, but not the 11-4, gene in human T cells by targeting transcription factors AP-1 and NF-AT. Cellular Immunology. 171(1):95-101; Paliogianni F. Hama N. Mavrothalassitis G J. Thyphronitis G. Boumpas D T. (1996) Signal requirements for interleukin 4 promoter activation in human T cells. Cellular Immunology. 168 (1):33-8; Davydov I V. Krammer P H. Li-Weber M. (1995) Nuclear factor-IL6 activates the human IL-4 promoter in T cells. Journal of Immunology. 155 (11):5273-9; Hama N. Paliogianni F. Fessler B J. Boumpas D T. (1995) Calcium/calmodulin-dependent protein kinase II downregulates both calcineurin and protein kinase C-mediated pathways for cytokine gene transcription in human T cells. Journal of Experimental Medicine. 181(3):1217-22.

### Example IV

These examples illustrate various ways which can use hNIP45 to screen for compounds which disrupt the interaction between hNIP45 and NFAT 1 (the human homologue of murine NFATp) and hence the production of IL-4.

Yeast Two-hybrid System(s)—(see, e.g., Example VIII)

Start with a Yeast Two-Hybrid System like the LexA system (or the GAL4 system).

Fuse hNFAT 1 coding sequence, for the DNA binding domain, in frame with the DNA binding moiety LexA (or GAL4).

Construct a HTPS strain by co-transforming a host yeast (e.g. EGY48) with the resulting construct together with an AD-hNIP45 (SEQ ID NO:2) fusion construct in which the hNIP45-interacting domain of NFAT1 is fused to a transcription activation domain like B42 (or VP16, GAL4, etc.), and a reporter construct which uses LacZ, LEU2, HIS3, etc. for measurable readout.

Prepare test compound collection with appropriate solvent in titer plates.

Seed test titer plates with log-phase HTPS strain culture, add dissolved test compounds, continue incubation of the test titer plates at 30° C. Use solvent only as a background control.

Measure the readout signal (depend on the reporter system actually to be used), record for comparison.

Those with significant inhibition (≧50%) of color development (if uses LacZ reporter), or of growth (if uses LEU2 or HIS3 reporters) can be accepted as modulators.

See, e.g.,. Banerjee A K. (1995) Interaction between the nucleocapsid protein and the phosphoprotein of human parainfluenza virus 3. Mapping of the interacting domains using a two-hybrid system. Journal of Biological Chemistry. 270 (21):12485-90; Wittmann S. Chatel H. Fortin M G. Laliberte J F. (1997) Interaction of the viral protein genome linked of turnip mosaic potyvirus with the translational eukaryotic initiation factor (iso) 4E of Arabidopsis thaliana using the yeast two-hybrid system. Virology. 234 (1):84-92; Beranger F. Aresta S. de Gunzburg J. Camonis J. (1997) Getting more from the two-hybrid system: N-terminal fusions to LexA are efficient and sensitive baits for two-hybrid studies. Nucleic Acids Research. 25 (10):2035-6; Zhu L. (1997) Yeast GAL4 two-hybrid system. A genetic system to identify proteins that interact with a target protein. Methods in Molecular Biology. 63:173-96, 1997.

### Example V

Use with the Scintillation Proximity Assay (SPA) system(s)

Over-express and purify hNIP45 and conjugate with the SPA beads.

Over-express and purify NFAT1 and label with ³H or ¹²⁵I, etc.

Aliquot the mix into titer plates.

Prepare test compound collection with appropriate solvent in titer plates.

Add dissolved compounds to the test titer plates.

Measure decrease of Scintillation signals and record.

Those with significant inhibition (≧50%) of readout signal are accepted as modulators.

See, Wiekowski M. Prosser D. Taremi S. Tsarbopoulos A. Jenh C H. Chou C C. Lundell D. Zavodny P. Narula S. (1997) Characterization of potential antagonists of human interleukin 5 demonstrates their cross-reactivity with receptors for interleukin 3 and granulocyte-macrophage colony-stimulating factor. European Journal of Biochemistry. 246(3):625-32; Hancock A A. Vodenlich A D. Maldonado C. Janis R. (1995) alpha2-adrenergic agonist-induced inhibition of cyclic AMP formation in transfected cell lines using a microtiter-based scintillation proximity assay. Journal of Receptor & Signal Transduction Research. 15(1-4):557-79; Kahl S D. Gygi T. Eichelberger L E. Manetta J V. (1996) A multiple-approach scintillation proximity assay to measure the association between Ras and Raf. Analytical Biochemistry. 243 (2):282-3; Sonatore L M. Wisniewski D. Frank L J. Cameron P M. Hermes J D. Marcy A l. Salowe S P. (1996) The utility of FK506-binding protein as a fusion partner in scintillation proximity assays: application to SH2 domains. Analytical Biochemistry. 240 (2):289-97; Lerner C G. Saiki AYC. (1996) Scintillation proximity assay for human DNA topoisomerase I using recombinant biotinyl-fusion protein produced in baculovirus-infected insect cells. Analytical Biochemistry. 240(2):185-96; Patel S. Harris A. O'Beirne G. Cook N D. Taylor C W. (1996) Kinetic analysis of inositol trisphosphate binding to pure inositol trisphosphate receptors using scintillation proximity assay. Biochemical & Biophysical Research Communications. 221 (3):821-5; Pachter J A. Zhang R. Mayer-Ezell R. (1995) Scintillation proximity assay to measure binding of soluble fibronectin to antibody-captured alpha 5 beta 1 integrin. Analytical Biochemistry. 230 (1):101-7; Carrick T A. Bingham B. Eppler C M. Baumbach W R. Zysk J R. (1995) A rapid and sensitive binding assay for growth hormone releasing factor. Endocrinology. 136 (10):4701-4.

### Example VI

Interaction Between hNIP45 and NFAT1

A yeast two hybrid system was employed as follows to demonstrate interaction between the human NIP45 homolog (hNIP45) described herein and human NFAT1 (hNFAT1).

A 879 bp DNA fragment, corresponding to positions 1418-2296 of hNFAT1 nucleic acid sequence (GenBank Accession number U43342), was PCR amplified via standard methods using Clontech human lymph node Marathon Ready cDNA as template. CLONTECH, Palo Alto, Calif. The resulting PCR product was cloned into the bait vector, plexA-BD (CLONTECH). The hybrid construct (plexA-BD-hNFAT1-BD) contains the coding region for 400-692 amino acid residues of hNFAT 1, which corresponds to the DNA binding region of the hNFAT 1 protein. The full length hNIP45H cNDA was cloned into prey vector pB42AD (CLONTECH). This hybrid construct (pB42AD-hNIP45H) contains the entire open reading frame of hNIP56H.

Yeast strain EGY48/p8op-lacZ (CLONTECH) was transformed with the plexA-BD-hNFAT1-BD plasmid. Transformants from SD/-UH plates did not yield any blue colonies on SD/-UH/X-Gal plates, indicating that the bait construct does not have intrinsic transcription activity. Yeast strain EGY/p8op-lacZ/plexA-BD-hNFAT1-BD was subsequently transformed with pB42AD-hNIP45H prey construct. Transformants from SD/-UHW plates were subsequently tested on a 4-plate test (SD/-UHW, X-gal vs. GR/-UHW, X-gal, and SD/-UHWL vs. GR/-UHWL). These transformants showed galactose dependent blue phenotype.

This experiment demonstrated that hNIP45 indeed interacts with hNFAT 1, and the interaction domain of hNFAT1 probably overlaps with its DNA binding domain. The yeast two hybrid system is also used for high throuput screening described herein. See FIG. 8 and FIG. 9.

### Example VII

Measuring LacZ Activity

The original method, and the method which describes Z buffer, is J. H. Miller, Experiments in Molecular Genetics, Cold Spring Harbor Press, 352 (1972). A similar method has been described for employment in yeast by Dohlman, H. G., et al., Mol. Cell. Biol. 15:3635 (1995), which is based upon a modification of the method of Guarente, L., Yeast Promoters and LacZ Fusions Designed to Study Expression of Cloned Genes in Yeast, Meth. Enzymol., 101:181 (1983).

Two Important Differences:

(1) CPRG is used rather than ONPG. LacZ appears to have a lower Km for CPRG than for ONPG. We have measured the Km as about 0.5 mM for CPRG. In assays SmM (10×Km) is used which we take to be approximately saturating.

(2) Chloroform is omitted from the Z buffer. This enables the reaction to be performed in plastic microtitre plates, and it enables the reaction to be performed by a single step addition of a buffer containing CPRG. In S. pombe, the presence of chloroform plus growth medium somehow inactivates lacZ- so that the cells have to be spun down and recovered if chloroform is used. SDS can satisfactorily replace chloroform.

The method is essentially as published (Dixon, G., et al., J. Steroid Biochem. Molec. Biol., 62:165 (1997)) except that 5 mM final CPRG is used instead of 0.5 mM final.

For a single substrate enzyme (B-gal is a hydrolase, so the substrates are H2O and e.g. CPRG, so technically is a two substrate enzyme, but water is present in saturating quantities and can be ignored), the rate of reaction is:

$$rate = kcat \frac{E . S}{Km + S}$$

where kcat is the catalytic constant and Km is the Michaelis constant. E is the enzyme concentration and S is the substrate concentration.

As S increases, the rate approaches kcat.E, or Vmax. So Vmax is proportional to the enzyme concentration, or E. Therefore we need to establish conditions for substrate which are saturating, and under which the rate will be proportional to the enzyme concentration.

| method | | |
|---|---|---|
| 10 × Z buffer (1 liter): | | |
| 600 mM | 85.2 g | Na2HPO4 |
| 400 mM | 62.4 g | NaH2PO4.2H20 |
| 100 mM | 7.5 g | KCl |
| 10 mM | 2.46 g | MgSO4.7H20 |

Dissolve in 500 mls H2O. Then add 2.7 mls b-mercaptoethanol and mix.

Adjust pH to 7.0 using 5M NaOH and make up to 1 liter.

Make up a 5×reaction buffer which contains 5×Z buffer, 25 mM CPRG and 0.1% SDS

Grow yeast cultures overnight. Dilute back to OD 0.05 to 0.1 next day and allow to continue growing. When they have reached an OD of about 1, make a set of dilutions in culture medium. Add 120 ul of each dilution to the wells of a microtitre plate. Start the reaction by addition of 30 ul reaction buffer and measure the initial rate of reaction (kinetic read over 10 minutes).

Two Ways to Calculate Units

(1) Measuring cell number as OD570 (initial read) in the plate. Subtract 0.035 from the OD570 initial read (this is the OD of an empty plate). Multiply the rate of OD570 change by 1.25 (the dilution factor; this allows for the alteration in path length upon dilution):

$$Units = \frac{( rate of OD 570 change ) \times 1.25}{( OD 570initial - 0.035 )}$$

(2) Measuring cell density in the spectrophotomoter at 600 nm. Since the path length is 1 cm, and in a 96-well plate the path length is 1 cm for 350 ul volume, correct for the path length difference by dividing volume by 350

$$Units = \frac{350 \times ( rate of OD 570 change ) \times 1.25}{Volume ( ul ) \times OD 600}$$

The number of cells is not linear with respect to the OD. Over the range 0-0.5 for OD 600, there is approximate linearity (OD of 0.5 , for a 10 mm path length, corresponds to 7×106 cells per ml; OD of 1 corresponds to 1.8×107; the higher the OD the more you underestimate the number of cells). Suggestion: measure the OD of the culture in a spec, and dilute to approximately 0.3.

If rate of change of OD570 is expressed as mOD/min, then the activity of Gal-mVP comes out as about 1500 units.

### Example VIII

A Yeast 2-hybrid Based HTS Procedure that Minimizes the Effect of Compound Toxicity for hNIP45/hNFAT1 Antagonists

The aim of the hNIP45/hNFAT1 high throughput screen is to identify inhibitors of hNIP45/hNFAT1 protein-protein interaction in order to block IL-4 gene activation involved in asthma and other immune disorders. hNIP45/hNFAT1 interaction has been characterized as important in the trans-activation of IL-4 gene, which has been recognized as the major modulator of asthma and other immune disorders. The screen is based on blocking hNIP45/hNFAT1 interaction will lead to a reduction of IL-4 gene activation. The role of these two proteins in IL-4 gene activation has been validated by co-transfection experiments with c-Maf. The interaction specificity of hNIP45 has been tested against control baits and that of and hNFAT1 has been tested against control preys including mNIP45.

This assay is a LexA yeast 2-hybrid based system and was made with hNIP45 (SEQ ID NO:2) and hNFAT1 ORF cDNA.

This described HTS procedure was specifically developed in order to minimize the effect of compound toxicity without using the protected reverse yeast 2-hybrid system. The compound toxicity was found to cause high false positive rate in the previous C2TA HTS experiments.

| | |
|---|---|
| Assay type: | Protein-protein interaction inhibition. |
| Test concentration: | 10⁻⁴M |
| DMSO concentration (final): | 1.0% |
| Assay temperature: | 30° C. |
| Incubation time: | 24 hours for induced expression and |
| | 2 hours for CPRG assay |

PROCEDURE

| | |
|---|---|
| I. | [DAY 0-1] Reagent preparation: |
| | CPRG (Chlorophenol red-b-D-galacopyranoside) from Boehringer, |
| | Mannheirm, Germany, 50 mM (100 ml). |
| | 20% SDS (100 ml), store at room temperature. |

10X Z buffer without b-2-ME (1 L):

| Reagent | Amount | unit | Final Conc. |
|---|---|---|---|
| Na₂HPO₄ | 85.2 | g | 600 mM |
| NaH₂PO₄.2H₂O | 64.2 | g | 400 mM |
| KCl | 7.5 | g | 100 mM |
| MgSO₄ | 246 | g | 10 mM |
| H₂O | 500 | ml | |
| Adjust pH to 7.0 with 5M NaOH. | | | |
| Adjust volume with H₂O to | 1 | L | |

| | Sterile H₂O, 4 L |
|---|---|
| | 1X “UHW” base medium (700 ml/1 L complete medium), 4 L. |
| | 10X (20%) dextrose (glucose), 1 L. |
| | 10X (20%) galactose, 1 L. |
| | 20X (20%) raffinose, 1 L. |
| II. | [DAY 1] Start the seed cultures: |

Complete 1X “SD/-UHW” medium (1 L):

| | | |
|---|---|---|
| “-UHW” base medium | 700 | ml |
| 10X(20%) glucose | 100 | ml |
| H₂O | 200 | ml |

Inoculate O/N cultures:

| | | | |
|---|---|---|---|
| Culture 1 | SD/-UHW, 50 ml | mNIP45/mNFTp | clone#1 |
| Culture 2 | SD/-UHW, 50 ml | mNIP45/mNFTp | clone#2 |
| Culture 3 | SD/-UHW, 50 ml | mNIP45/mNFTp | clone#3 |
| Culture 4 | SD/-UHW, 50 ml | hNIP4S/hNFT1 | clone#1 |
| Culture 5 | SD/-UHW, 50 ml | hNIP45/hNFT1 | clone#2 |
| Culture 6 | SD/-UHW, 50 ml | hNIP45/hNFT1 | clone#3 |

| | => Grow O/N at 30° C. with agitation. |
|---|---|
| III. | [DAY 2] Induction for prey expression (from GAL1 promoter): |

Prepare 1X “-UHW” base medium (1 L):

| | | |
|---|---|---|
| “-UHW” base medium | 700 | ml |
| H₂O | 300 | ml |

Complete 1X “-UHW” with 2X GR medium (1 L):

| | | |
|---|---|---|
| “-UHW” base medium | 700 | ml |
| 10X (20%) galactose | 200 | ml |
| 20X (20%) raffinose | 100 | ml |

aliquot 80 ml 2X GR medium to each well in the titer plates filled
with 40 ml compounds to be screened.
aliquot 80 ml 2X GR medium to each well in an additional titer plate
filled with 40 ml water as a control plate for the screen.
Prepare the cells for assay:
=> pellet, decant and rinse the cells with sterile H₂O twice.
=> re-suspend the cells with 1X “-UHW” base medium till
OD600 = 1.0, record vol.

| | | Vol of 1X “UHW” base | |
|---|---|---|---|
| O/N cultures | Y2 partners | medium to OD600 = 1.0 | Total Vol |
| 4 | hNIP45/hNFT1 | | |
| 5 | hNIP45/hNFT1 | | |
| 6 | hNIP45/hNFT1 | | |

| | pool equal vol of the resuspended cultures #4-6 together. |
|---|---|
| | aliquot 40 ml cells to each well in the test and control titer plates. |
| | => mix, read/record (on-line) OD600, seal/cover, incubate at |
| | 30° C. for 24 hours. |
| IV. | [DAY 3] Liquid b-gal assay: |

Preparing 1X b-gal assay control solution (10 ml):

| | | |
|---|---|---|
| 10X Z buffer | 5 | ml |
| H₂O | 5 | ml |
| SDS, 20% | 500 | μl |
| β-mercaptoethanol | 27 | μl |

Preparing 1X b-gal assay substrate solution (1 L):

| | | |
|---|---|---|
| 10X X buffer | 500 | ml |
| H₂O | 447.3 | ml |
| CPRG, 50 mM | 50 | ml |
| SDS, 20% | 5 | ml |
| β-mercaptoethanol | 2.7 | ml |

b-gal assay:
=> add 40 ml of the 1X control solution to the wells in the control
titer plate.
=> add 40 ml of the 1X substrate solution to the wells in the test
titer plates.
=> mix, read/record (on-line) OD600 and OD570.
=> incubate at 30° C., read/record (on-line) OD600 and OD570
after 2 hours.
Calculations:
b-gal activityₛₐₘₚₗₑ = OD570_{t=2 hrs} − OD570_{t=0 hrs}
$Toxicity {index}_{sample} = \frac{{OD600}_{t = 2 hrs} [ sample ]}{{OD600 }_{t = 2 hrs} [ control , no compound ]}$
No Tox b-gal activityₛₐₘₚₗₑ = b-gal activityₛₐₘₚₗₑ/Toxicity indexₛₐₘₚₗₑ
$Inhibition {index}_{sample} - 1 - \frac{No Tox b \text{-} gal {activity}_{sample}}{No Tox b \text{-} gal {activity}_{control}}$
Data processing:
Organize data and calculate the inhibition index of each screened
compound.
Focus on the compounds with an inhibition index greater than 0.5
(50% inhibition).
Subject the IPS-filtered compounds to secondary effector-gene (IL-4)
sensitive assays
with activated human PBMC.

→ See FIG. 10

All publications and patents mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

What is claimed is

1. A purified polynucleotide comprising a nucleic acid sequence encoding the polypeptide having the sequence substantially as depicted in SEQ ID NO:3 or a biologically active fragment thereof.

2. The polynucleotide of claim 1 wherein the polynucleotide sequence comprises the sequence substantially as depicted in SEQ ID NO:2.

3. An expression vector comprising the polynucleotide of claim 1.

4. An antisense molecule comprising the complement of the polynucleotide of claim 2 or a biologically-effective portion thereof.

5. A host cell transformed with the expression vector of claim 3.

6. A purified polypeptide comprising the amino acid sequence substantially as depicted in SEQ ID NO:3.

7. An antibody specific for the polypeptide of claim 6.

8. A method for producing cells which express a biologically active polypeptide substantially as depicted in SEQ ID NO:3, said method comprising
a) culturing a host cell according to claim 5 under conditions suitable for the expression of said polypeptide.

9. A method for producing a polypeptide having the amino acid sequence substantially as depicted in SEQ ID NO:3, said method comprising the steps of:
a) culturing a host cell according to claim 5 under conditions suitable for the expression of said polypeptide, and
b) recovering said polypeptide from the host cell culture.

10. A method of identifying compounds that modulate the biological activity of human NIP45, comprising:
(a) combining a candidate compound modulator of human NIP45 biological activity with a NIP45 polypeptide comprising the sequence substantially as depicted in SEQ ID NO:3, and
(b) measuring an effect of the candidate compound modulator on the biological activity.

11. A method of identifying compounds that modulate the biological activity of human NIP45, comprising:
(a) combining a candidate compound modulator of human NIP45 biological activity with a host-cell expressing a NIP45 polypeptide comprising the sequence substantially as depicted in SEQ ID NO:3, and
(b) measuring an effect of the candidate compound modulator on the biological activity.

12. A method of identifying compounds that modulate the transcriptional activation of IL-4, comprising:
(a) combining a candidate compound modulator of transcriptional activation of IL-4 with a polypeptide of a human trans-activator having the sequence substantially as depicted in SEQ ID NO:3, and an IL-4 cistron, and
(b) measuring an effect of the candidate compound modulator on the transcriptional activation of the IL-4 cistron.

13. A method of identifying compounds that modulate the transcriptional activation of IL-4, comprising:
(a) combining a candidate compound modulator of transcriptional activation of IL-4 with a host-cell comprising an IL-4 cistron and expressing a polypeptide of a human trans-activator having the sequence substantially as depicted in SEQ ID NO:3, and
(b) measuring an effect of the candidate compound modulator on the transcriptional activation of the IL-4 cistron.

14. A compound that modulates the biological activity of a human NIP45 identified by the method of claim 10.

15. A compound that modulates the biological activity of a human NIP45 identified by the method of claim 11.

16. A compound that modulates the transcriptional activation of IL-4 identified by the method of claim 12.

17. A compound that modulates the transcriptional activation of IL-4 identified by the method of claim 13.

18. A pharmaceutical composition comprising a compound that modulates the biological activity of a human NIP45 according to claim 14.

19. A pharmaceutical composition comprising a compound that modulates the biological activity of a human NIP45 according to claim 15.

20. A pharmaceutical composition comprising a compound that modulates the transcriptional activation of IL-4 according to claim 16.

21. A pharmaceutical composition comprising a compound that modulates the transcriptional activation of IL-4 according to claim 17.

22. A method of treatment of a patient in need of such treatment for a condition which is mediated by the biological activity of human NIP45, comprising administration of a modulating compound according to claim 14.

23. A method of treatment of a patient in need of such treatment for a condition which is mediated by the biological activity of human NIP45, comprising administration of a modulating compound according to claim 15.

24. A method of treatment of a patient in need of such treatment for a condition which is mediated by the biological activity of IL-4, comprising administration of a modulating compound according to claim 16.

25. A method of treatment of a patient in need of such treatment for a condition which is mediated by the biological activity of IL-4, comprising administration of a modulating compound according to claim 17.

26. A method for inhibiting the expression of a NIP45 in a cell comprising administering an effective amount of an antisense molecule according to claim 4 to said cell.

27. A method for modulating the expression of IL-4 in a cell comprising administering an effective amount of an antisense molecule according to claim 4 to said cell.

28. A diagnostic composition for the identification of a polypeptide sequence comprising the amino acid sequence substantially as depicted in SEQ ID NO:3, comprising the antibody of claim 7.

29. A diagnostic composition for the identification of a polynucleotide sequence comprising the sequence substantially as depicted in SEQ ID NO:2 or a fragment indicative thereof, comprising PCR primers derived from SEQ ID NO:1.


SEQUENCE LISTING (4 sequences)

SEQ ID NO: 1 - DNA, 2576 bp, Homo sapiens
ggaaagtgtgccatggcggagcctgtggggaagcggggccgctggtccggaggtagcggtgccggccgagggggtcggggcggctggggcggtcggggccggcgtcctcgggcccagcggtctccatcccggggcacgctggacgtagtgtctgtggacttggtcaccgacagcgatgaggaaattctggaggtcgccaccgctcgcggtgccgcggacgaggttgaggtggagcccccggagcccccggggccggtcgcgtcccgggataacagcaacagtgacagcgaaggggaggacaggcggcccgcaggacccccgcgggagccggtcaggcggcggcggcggctggtgctggatccgggggaggcgccgctggttccggtgtactcggggaaggttaaaagcagccttcgccttatcccagatgatctatccctcctgaaactctaccctccaggggatgaggaagaggcagagctggcagattcgagtggtctctaccatgagggctccccatcaccaggctctccctggaagacaaagctgaggactaaggataaagaagagaagaaaaagacagagtttctggatctggacaactctcctctgtccccaccttcaccaaggaccaaaagcagaacgcatactcgggcactcaagaagttaagtgaggtgaacaagcgcctccaggatctccgttcctgtctgagccccaagccacctcagggtcaagagcaacagggccaagaggatgaagtggtcttggtggaagggcccaccctcccagagaccccccgactcttcccactcaaaatccgttgccgggctgacctggtcagattgcccctcaggatgtcggagcccctgcagagtgtggtggaccacatggccacccaccttggggtgtccccaagcaggatccttttgctttttggagagacagagctatcacctactgccactcccaggaccctaaagctcggagtggctgacatcattgactgtgtggtactaacaagttctccagaggccacagagacgtcccaacagctccagctccgggtgcagggaaaggagaaacaccagacactggaagtctcactgtctcgagattcccctctaaagaccctcatgtcccactatgaggaggccatgggactgtcgggacggaagctctccttcttctttgatgggacaaagctttcaggcagggagctgccagctgacctgggcatggaatctggggacctcattgaggtctggggctgacaccccactccctgtttgacggcccagcctggacttggggagaatgactttcccttttttgccccataagggctagcataagctgaggtagaacttatctttaagctgcagcaaaatcaaggagtgacttttgtcccctctcctgttgaccctggtttagagccgttaaccacttggtgagttatgtgggtgttgttgccctgggtggcctgtggctccgtccacaagtcatgctgagttttgcagcctctgtgacttggagatgtcccttcacccctcccctttcaccaccatcctcttttcctcatggaaatgtctgctttatgaaactatgcacatattgaaagtgagttgaaacaaatgagggttgggtaggagcttccaggcctgggatttacaccacgcctagcccagcagaggccttagtcccatttggggcttgggagtgacatttgcttgaggcttatacactggtgtggttgcctggcttgcaggaaatgaccaagctcacacatgctggctgaagcgtaagcagacaactgaggtactcttttgaaggatgaaggtggtggattctcagccctgggggtcttcctcacctgaggacccttcagagccaccctttctagtttgcatttcctggtgcacacatttaaggcataacagcacattcatccctttggtttgggatctcaggaatacagtcccatgcaaagattctctggttttatggcttttttccctttctttacaccatcctctcccataagcacccatgtctttgaatatgaatgtatttgtaaaataccacgtttcatgtgtgaatatgtgcttttactgtacatagtgctattgtgcaataggtcttatgctgttttcactcaatgtgtgctaagatctagccccattgactcttctagaaatgcagtattgctttgacctgccatgtggcactccacaatgtcaattgcagtttacacacattgcctaaagtgggggacacctgggtgcccctgaccccttggcaccggatacaggccacgataaacatcctttcgtgtgttcccttctgtgcttgtgtggcatgtgtacccaggatgggcctataggtcacagaggtcagtttctctttggttttccagattttctttagaacggtgactgaccctcctacttgaggccgcccttttctccttatccttgccagcacttgtattgccagactacctaatttttgccagtctcatgggtagatagtggtgcagtgctttaacatacattcatctgatcagcattaatttggg

SEQ ID NO: 2 - DNA, 1260 bp, Homo sapiens
atggcggagcctgtggggaagcggggccgctggtccggaggtagcggtgccggccgagggggtcggggcggctggggcggtcggggccggcgtcctcgggcccagcggtctccatcccggggcacgctggacgtagtgtctgtggacttggtcaccgacagcgatgaggaaattctggaggtcgccaccgctcgcggtgccgcggacgaggttgaggtggagcccccggagcccccggggccggtcgcgtcccgggataacagcaacagtgacagcgaaggggaggacaggcggcccgcaggacccccgcgggagccggtcaggcggcggcggcggctggtgctggatccgggggaggcgccgctggttccggtgtactcggggaaggttaaaagcagccttcgccttatcccagatgatctatccctcctgaaactctaccctccaggggatgaggaagaggcagagctggcagattcgagtggtctctaccatgagggctccccatcaccaggctctccctggaagacaaagctgaggactaaggataaagaagagaagaaaaagacagagtttctggatctggacaactctcctctgtccccaccttcaccaaggaccaaaagcagaacgcatactcgggcactcaagaagttaagtgaggtgaacaagcgcctccaggatctccgttcctgtctgagccccaagccacctcagggtcaagagcaacagggccaagaggatgaagtggtcttggtggaagggcccaccctcccagagaccccccgactcttcccactcaaaatccgttgccgggctgacctggtcagattgcccctcaggatgtcggagcccctgcagagtgtggtggaccacatggccacccaccttggggtgtccccaagcaggatccttttgctttttggagagacagagctatcacctactgccactcccaggaccctaaagctcggagtggctgacatcattgactgtgtggtactaacaagttctccagaggccacagagacgtcccaacagctccagctccgggtgcagggaaaggagaaacaccagacactggaagtctcactgtctcgagattcccctctaaagaccctcatgtcccactatgaggaggccatgggactgtcgggacggaagctctccttcttctttgatgggacaaagctttcaggcagggagctgccagctgacctgggcatggaatctggggacctcattgaggtctggggctga

SEQ ID NO: 3 - protein, 419 aa, Homo sapiens
MAEPVGKRGRWSGGSGAGRGGRGGWGGRGRRPRAQRSPSRGTLDVVSVDLVTDSDEEILEVATARGAADEVEVEPPEPPGPVASRDNSNSDSEGEDRRPAGPPREPVRRRRRLVLDPGEAPLVPVYSGKVKSSLRLIPDDLSLLKLYPPGDEEEAELADSSGLYHEGSPSPGSPWKTKLRTKDKEEKKKTEFLDLDNSPLSPPSPRTKSRTHTRALKKLSEVNKRLQDLRSCLSPKPPQGQEQQGQEDEVVLVEGPTLPETPRLFPLKIRCRADLVRLPLRMSEPLQSVVDHMATHLGVSPSRILLLFGETELSPTATPRTLKLGVADIIDCVVLTSSPEATETSQQLQLRVQGKEKHQTLEVSLSRDSPLKTLMSHYEEAMGLSGRKLSFFFDGTKLSGRELPADLGMESGDLIEVWG

SEQ ID NO: 4 - protein, 412 aa, Mus musculus
MAEPLRGRGPRSRGGRGARRARGARGRCPRARQSPARLIPDTVLVDLVSDSDEEVLEVADPVEVPVARLPAPAKPEQDSDSDSEGAAEGPAGAPRTLVRRRRRRLLDPGEAPVVPVYSGKVQSSLNLIPDNSSLLKLCPSEPEDEADLTNSGSSPSEDDALPSGSPWRKKLRKKCEKEEKKMEEFPDQDISPLPQPSSRNKSRKHTEALQKLREVNKRLQDLRSCLSPKQHQSPALQSTDDEVVLVEGPVLPQSSRLFTLKIRCRADLVRLPVRMSEPLQNVVDHMANHLGVSPNRILLLFGESELSPTATPSTLKLGVADIIDCVVLASSSEATETSQELRLRVQGKEKHQMLEISLSPDSPLKVLMSHYEEAMGLSGHKLSFFFDGTKLSGKELPADLGLESGDLIEVWG